(19) United States
(12) Patent Application Publication
(10) Pub. No.: US 2011/0289625 A1
(43) Pub. Date: Nov. 24, 2011

(54) ALTERED LEAF MORPHOLOGY AND ENHANCED AGRONOMIC PROPERTIES IN PLANTS

(76) Inventors: Rujin Chen, Ardmore, OK (US); Jianghua Chen, Ardmore, OK (US); Liangfa Ge, Ardmore, OK (US); Yasuhiro Ishiga, Ardmore, OK (US); Kirankumar Mysore, Ardmore, OK (US); Srinivasa Rao Uppalapati, Lone Grove, OK (US)

(21) Appl. No.: 13/111,796

(22) Filed: May 19, 2011

Related U.S. Application Data
(60) Provisional application No. 61/346,373, filed on May 19, 2010.

(51) Int. Cl. A01H 5/00; A01H 5/02; A01H 5/04; A01H 5/12; A23K 1/14; C12N 15/113; C12N 5/10; C12N 15/82; C07C 29/74; A23K 3/02; A01H 5/10; C12N 5/04

(52) U.S. Cl. 800/279; 800/298; 800/320; 800/312; 800/320.1; 800/301; 435/410; 536/24.1; 435/419; 800/278; 800/281; 568/913; 426/54; 426/665

(57) ABSTRACT

The invention provides methods for enhancing agronomic properties in plants by down-regulation of a PALM transcription factor. Nucleic acid constructs for down-regulation of PALM are described. Transgenic plants are provided that comprise increased leafy tissue and increased disease resistance. Plants described herein may be used, for example, as improved forage crops or in biofuel production.

This application claims the priority of U.S. Provisional Appl. Ser. No. 61/346,373, filed May 19, 2010, the entire disclosure of which is incorporated herein by reference.

### STATEMENT REGARDING FEDERALLY SPONSORED RESEARCH OR DEVELOPMENT

This invention was made with Government support under DBI 0703285 and EPS 0814361 awarded by the National Science Foundation. The Government has certain rights in the invention.

### INCORPORATION OF SEQUENCE LISTING

The sequence listing that is contained in the file named “NBLE073US_ST25.txt”, which is 61,510 bytes (measured in MS-WINDOWS) and was created on May 19, 2011, is filed herewith by electronic submission and incorporated herein by reference

### BACKGROUND OF THE INVENTION

1. Field of the Invention

The present invention relates generally to the field of agriculture and plant genetics. More particularly, it concerns genetically modified plants comprising enhanced agronomic properties including improved disease resistance.

2. Description of Related Art

Genetic modification of plants has, in combination with conventional breeding programs, led to significant increases in agricultural yield over the last decades. However, most genetically modified plants are selected for a single agronomic trait often by expression of a single enzyme coding sequence (e.g., enzymes that provide herbicide resistance). To date, there has been little progress in developing plants that comprise modified gene expression profiles and thereby exhibit a variety of characteristics that are of agronomic interest.

### SUMMARY OF THE INVENTION

In a first embodiment there is provided a plant comprising a down-regulated PALM transcription factor wherein the plant exhibits an enhanced agronomic property. In certain aspects, a plant comprising a down-regulated PALM transcription factor comprises altered leaf morphology. For example, a plant may comprise compound leaves with additional leaflets, such as pentafoliate leaves. In further aspects, a plant according to the invention comprises an enhanced agronomic property, such as increased disease resistance, increased nutritional content or increased leafy tissue.

As used herein, the term PALM transcription factor refers to the PALM1 polypeptide from M. truncatula (SEQ ID NO: 70; GenBank Accession HM038482) and variants, homologs and orthologs thereof. For example, the PALM transcription factor may be a PALM polypeptide from M. sativa (SEQ ID NO: 72), Glycine max (PALM1; SEQ ID NO: 74) or (PALM2; SEQ ID NO: 76), Lotus japonicus (SEQ ID NO: 78), Arabidopsis thaliana (SEQ ID NO: 80), Vitis vinifera (SEQ ID NO: 82), Arabidopsis lyrata (SEQ ID NO: 84), Cucumis sativus (SEQ ID NO: 86), Manihot esculenta (SEQ ID NO: 88), Mimulus guttatus (SEQ ID NO: 90), Populus trichocarpa (SEQ ID NO: 92), Ricinus communis (SEQ ID NO: 94) or Carica Papaya (SEQ ID NO: 96). A homolog may be defined, for instance, as a gene encoding a polypeptide having at least 60%, 70%, 80%, 85%, 90%, 95%, 98% or greater amino acid identity to SEQ ID NOs: 70, 72, 74, 76, 78, 80, 82, 84, 86, 88, 90, 92, 94 or 96.

In one embodiment, a plant cell is provided that comprises a down-regulated PALM transcription factor. For example, the plant cell can comprise a genomic PALM gene comprising a mutation that disrupts the gene by decreasing PALM expression, by abrogating expression entirely or by rendering the gene product non-functional. The mutation may be a point mutation, an insertion or a deletion and the mutation may be located in a protein coding region or non-coding portion to the PALM gene (e.g., in the PALM promoter region). Mutations in a PALM gene can be accomplished by any of the methods well known to those in the art including random mutagenesis methods such as irradiation, random DNA integration (e.g., via a transposon) or by using a chemical mutagen. Moreover, in certain aspects, a PALM gene may be mutated using a site-directed mutagenesis approach such as by using homologous recombination vector. Further detailed methods for inducing mutations in plant genes are provided below.

In a further embodiment, a plant cell is provided comprising a DNA molecule capable of expressing a nucleic acid sequence complementary to all or a portion of a PALM gene sequence or a PALM messenger RNA (mRNA). Thus, in some aspects, a transgenic plant may comprise DNA that expresses an antisense, RNAi or miRNA molecule for down-regulation of a PALM transcription factor. For example, a transgenic plant can comprise a promoter which expresses a sequence complimentary to all or a portion of a PALM sequence from the plant. In certain specific embodiments, a transgenic plant comprises a nucleic acid molecule capable of expressing an nucleic acid sequence complementary to all or a portion of a PALM mRNA from M. truncatula (SEQ ID NO: 69), M. sativa (SEQ ID NO: 71), Glycine max (PALM1; SEQ ID NO: 73) or (PALM2; SEQ ID NO: 75), Lotus japonicus (SEQ ID NO: 77), Arabidopsis thaliana (SEQ ID NO: 79), Vitis vinifera (SEQ ID NO: 81), Arabidopsis lyrata (SEQ ID NO: 83), Cucumis sativus (SEQ ID NO: 85), Manihot esculenta (SEQ ID NO: 87), Mimulus guttatus (SEQ ID NO: 89), Populus trichocarpa (SEQ ID NO: 91), Ricinus comunis (SEQ ID NO: 93) or Carica Papaya (SEQ ID NO: 95). Moreover, in certain aspects, the DNA that down regulates PALM may comprise a tissue specific or inducible promoter operably linked to the nucleic acid sequence complimentary to all or part of a PALM gene or mRNA. In some cases, the promoter sequence is selected from the group consisting of a developmentally-regulated, organelle-specific, inducible, tissue-specific, constitutive, cell-specific, seed specific or germination-specific promoter.

A variety of plants can be modified in accordance with the instant disclosure. For example, in some aspects, a plant comprising a down-regulated PALM transcription factor may be a forage plant, a biofuel crop, a legume, or an industrial plant. For example, a forage plant may be a forage soybean, alfalfa, clover, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass or reed canarygrass plant. In certain aspects, a plant is a biofuel crop including, but not limited to, switchgrass (Panicum virgatum), giant reed (Arundo donax), reed canarygrass (Phalaris arundinacea), Miscanthus×giganteus, Miscanthus sp., sericea lespedeza (Lespedeza cuneata), corn, sugarcane, sorghum, millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass or poplar. Legume plants for use according to the instant disclose include, but are not limited to, plants from inverted repeat lacking clade (IRLC) such as the garden pea (Pisum sativum) and alfalfa (Medicago sativa). A legume plant may also be a soybean or peanut, or a pulse such as a Phaseolus vulgaris, Phaseolus lunatus, Vigna angularis, Vigna radiata, Vigna mungo, Phaseolus coccineus, Vigna umbellata, Vigna acontifolia, Phaseolus acutifolius, Vicia faba, Pisum sativum, Cicer arietinum, Vigna unguiculata, Cajanus cajan, Lens culinaris, Vigna subterranea, Vicia sativa or Lupinus spp. plant. In still further aspects a plant according to the invention may be a Vitis vinifera, Arabidopsis lyrata, Cucumis sativus, Manihot esculenta, Mimulus guttatus, Populus trichocarpa, Ricinus comunis or Carica Papaya.

In still further aspects, there is provided a part of a plant described herein such as a protoplast, cell, meristem, root, pistil, anther, flower, leaf, seed, embryo, stalk or petiole.

In another embodiment, a transgenic or mutated plant according to the invention may be further defined as an R0 plant, or as a progeny plant of any generation of an R0 plant, wherein the plant has inherited the selected DNA or mutation from the R0 plant. Moreover, in certain aspects, the a progeny plant as described herein may be defined as a progeny plant that has been crossed with a second plant, such as a variety with increased disease resistance or enhanced yield. In other embodiments, the invention comprises a seed of a plant wherein the seed comprises a mutation or selected DNA that down-regulates a PALM transcription factor. A transgenic cell of such a plant also comprises an embodiment of the invention.

In still a further embodiment, there is provided a polynucleotide molecule comprising a nucleic acid sequence selected from the group consisting of: a nucleic acid sequence that hybridizes to the nucleic acid sequence of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95, under stringent hybridization conditions (e.g., conditions of 1×SSC and 65° C.); (b) a nucleic acid comprising the sequence complementary to SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95 or a fragment thereof; and (c) a nucleic acid sequence exhibiting at least 80% sequence identity to a complement of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95; wherein the nucleic acid sequence is operably linked to a heterologous promoter sequence and wherein expression of the nucleic acid molecule in a plant cell down-regulates expression of a PALM transcription factor. In some aspects, a polynucleotide molecule provided comprises a nucleic acid sequence having at least 85%, 90%, 95%, 96%, 97%, 98% or 99% sequence identity to the full compliment of the nucleic acid sequence of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95. In further aspects, a polynucleotide molecule comprises a nucleic acid sequence complementary to at least 17, 18, 19, 20, 21, 25 or 30 nucleotides of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95; wherein the nucleic acid sequence is operably linked to a heterologous promoter sequence and wherein expression of the nucleic acid molecule in a plant cell down-regulates expression of a PALM transcription factor.

In yet a further embodiment, there is provided a polynucleotide molecule comprising a nucleic acid sequence selected from the group consisting of: a nucleic acid sequence that hybridizes to the nucleic acid sequence complementary to the sequence of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95, under stringent hybridization conditions (e.g., conditions of 1X SSC and 65° C.); (b) a nucleic acid comprising the sequence of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95 or a fragment thereof; (c) a nucleic acid sequence exhibiting at least 80% sequence identity to SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95; and (d) a nucleic acid sequence encoding polypeptide at least 90% (e.g., at least 95%, 97%, 98%, or 99%) identical to SEQ ID NO: 70; SEQ ID NO: 72; SEQ ID NO: 74; SEQ ID NO: 76; SEQ ID NO: 78; SEQ ID NO: 80; SEQ ID NO: 82; SEQ ID NO: 84; SEQ ID NO: 86; SEQ ID NO: 88; SEQ ID NO: 90; SEQ ID NO: 92; SEQ ID NO: 94; or SEQ ID NO: 96; wherein the nucleic acid sequence is operably linked to a heterologous promoter sequence and wherein expression of the nucleic acid molecule in a plant cell down-regulates expression from a SGL1 promoter sequence.

In further embodiments a transgenic plant, plant part or plant cell comprising a nucleic acid molecule as described herein is provided. For example, in certain aspects, nucleic acid molecules are provided that down-regulate PALM expression. Plants and plant parts comprising a down-regulated PALM may, in certain aspects, be defined as comprising in creased leaf:stem biomass. In certain aspects, such plants may be used for forage or a as feedstock for biofuel production.

Moreover, there is provided herein a method of increasing disease resistance in a plant comprising down-regulating a PALM transcription factor in the plant. For example, a method of increasing disease resistance can comprise expressing a nucleic acid molecule in a plant comprising a sequence complementary to a coding sequence for a PALM transcription factor thereby down-regulating expression of a PALM transcription factor. In certain aspects, a method for increasing resistance to a fungal pathogen, such as a rust pathogen (e.g., switchgrass rust or Asian soybean rust) is provided. For example, the fungal pathogen may be a Phakopsora pachyrhizi, Puccinia emaculata or Colletotrichum trifolii pathogen. In some embodiments, reduced expression of a PALM transcription factor in a plant, such as by down-regulating the expression of a PALM transcription factor in a plant, results in reduced formation of a pre-infection structure by a fungal plant pathogen contacting the plant.

In still a further embodiment, there is provided a method for increasing the digestibility of a forage crop comprising down-regulating a PALM transcription factor in the plant. For example, in certain aspects, plants described herein comprise increased leafy tissue mass and have enhanced digestibility. In some cases such plants or parts thereof may be used for livestock forage or in the manufacture of a livestock feed.

In yet a further embodiment there is provided a method of increasing the leaf:stem ratio of a plant comprising expressing a nucleic acid molecule in a plant comprising a sequence complementary to a coding sequence for a PALM transcription factor. Plants provided herein comprising increased leafy tissue (increased leaf:stem ratio) may, in certain aspects, be used in the manufacture of biofuel feedstock (e.g., ethanol and biodiesel) materials.

In still a further aspect, the instant disclosure provides a method of altering the leaf wax content of a plant comprising down-regulating a PALM transcription factor in the plant.

In still a further embodiment, there is provided a method for the manufacture of a commercial product comprising obtaining a plant or plant part comprising a mutation or a selected DNA that down-regulates a PALM transcription factor and producing a commercial product therefrom. For example, a plant or plant part described herein can be manufactured into products such as, paper, paper pulp, ethanol, biodiesel, silage, animal feed or fermentable biofuel feedstock.

In yet another aspect, the invention provides a method of producing ethanol comprising: (a) obtaining a plant of a biofuel crop species comprising a selected DNA that down-regulates a PALM transcription factor in the plant wherein the plant exhibits an increase in leafy tissue; (b) treating tissue from the plant to render carbohydrates in the tissue fermentable; and (c) fermenting the carbohydrates to produce ethanol.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan however these terms may be used interchangeably with “comprise” or “comprising” respectively.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1a-b: Medicago truncatula palm1-1 mutant exhibits altered leaf form. FIG. 1a, measurements of the petiole length of compound leaves on the fifth node of 6-week-old wild type and palm1-1 mutant plants. FIG. 1b, Measurements of the rachis length of compound leaves. Shown are means±s.e. (n=10).

FIG. 2a-d: Map-based cloning and characterization of PALM1. FIG. 2a, palm1 was mapped to contig 77 of chromosome 5 closely linked to the CR932963-SSR1 marker. Top showing markers that co-segregate with palm1; Bottom showing the number of recombinants. FIG. 2b, bacterial artificial chromosome clones in the region. FIG. 2c, deletion borders identified in palm1-1 and palm1-2 mutants using chromosomal walking. FIG. 2d, eight open reading frames (ORFs) annotated within the deleted region in palm1-1 and palm1-2 alleles. Solid boxes/vertical lines denoting exons and horizontal lines denoting introns.

FIG. 3: Amino acid sequence alignments of Medicago truncatula PALM1 (MtPALM) and its homologues from alfalfa (M. sativa), Lotus japonicus, soybean (Glycine max) and Arabidopsis thaliana. Boxed amino acids denote conserved residues. The Cys(2)His(2) zinc finger DNA-binding domain and the EAR transcriptional repressor domain are underlined.

FIG. 4: In silico analysis of tissue-specific expression of PALM1. Tissue-specific expression pattern of PALM1 was analyzed using Affymetrix GeneChip Medicago Genome Array-based Medicago Gene Expression Atlas (available at: bioinfo.noble.org/gene-atlas/v2/). Shown are the expression profiles of PALM1 in major organs including roots, nodules, stems, petioles, leaves, vegetative buds, flowers, seeds and seed pods with detailed developmental time-series for nodules and seeds.

FIG. 5a-c: Electrophoretic mobility shift assay (EMSA). FIG. 5a, a schematic drawing of the SGL1 promoter including F1, F2 and F3 sequences upstream from the translation initiation codon and a series of deletion fragments of the F2 sequence. FIG. 5b, EMSA of the F2 deletion sequences labeled with biotin in the presence and absence of purified His-tagged PALM1. FIG. 5a, EMSA of the F2-3 sequence. Tested were the unlabeled F2-3 sequence in 10-, 20- and 50-fold excess relative to the biotin-labeled sequence as specific competitors (indicated by +, ++, and +++), unlabeled F3-1 sequence in 50-fold excess as a non-specific (NS) competitor, and His-tagged TEV (tobacco etch virus protease), an unrelated protein as a negative control. Arrows indicating shifted bands.

FIG. 6a-c: M. truncatula irg1 mutant shows resistance to a broad-spectrum of rust pathogens including ASR. Graphs demonstrate that infection of irg1 mutant lines supported less spore adhesion (FIG. 6a), less formation of infection structures, appressoria (FIG. 6b) and resulted in low frequency of penetration (FIG. 6c).

FIG. 7: Accumulation of defense-related gene transcripts during interactions of P. pachyrhizi with M. truncatula wild-type (R108) and irg1 mutant. Expression profiles for selected genes in phenylpropanoid pathway including, PAL, phenyl alanine lyase; CHR, chalcone reductase; CHI, chalcone isomerase; CHS, chalcone synthase; IFS, isoflavone synthase and IFR, isoflavone reductase and pathogenesis-related genes (PR3 and PR10). Ten micrograms of total RNA was loaded and the ethidium bromide staining of rRNA shows equal quality and quantity of each samples.

FIG. 8: M. truncatula irg1 mutant lines shows resistance to Colletotrichum trifolii. Graph shows the percentage infection structures formed on Wild-type (R108) and irg1 (irg) mutants. Approximately, 100 spores were inoculated per spot and the infection structures were evaluated 24 hours post-inoculation.

FIG. 9a-c: Leaf phenotype of four-week old wild-type R108 and irg1-1 plants of M. truncatula. The adaxial (FIG. 9a) and abaxial (FIG. 9b) leaves of M. truncatula R108 and irg1. FIG. 9c: The abaxial leaf surface of M. truncatula R108 and irg1-1 under light.

FIG. 10a-b: Rust germ-tube differentiation on multiple mutant alleles of irg1. FIG. 10a: schematic showing the location of Tnt1 insertion in the PALM1/IRG1 exon of the different mutant alleles and corresponding line numbers used in this study. FIG. 10b: multiple alleles of Medicago truncatula irg1 mutant lines inhibit differentiation of pre-infection structure formation of Phakopsora pachyrhizi on abaxial surface of leaves.

FIG. 11 Medicago truncatula irg1-1 mutants inhibit switchgrass rust differentiation. Epifluorescence (FIG. 11a) and confocal (FIG. 11b) micrographs of WGA-Alexa Fluor® 488 stained germ-tubes of Puccinia emaculata on abaxial leaf surfaces of M. truncatula wild-type R108 and inhibitor of rust germ-tube differentiation (irg1-1) mutant of M. truncatula. P. emaculata spores germinated and formed long germ-tubes (LG) within 72 hpi on wild-type R108, and on irg1-1, spores germinated but formed very short germ-tubes (SG). Samples from an independent experiment were used for confocal imaging. Scale bar=100 μm.

FIG. 12: Multiple alleles of Medicago irg1 mutant inhibit differentiation of pre-infection structure formation of Puccinia emaculata on abaxial surface of leaves.

FIG. 13: Expression profiles for selected genes in phenylpropanoid pathway (PAL, CHS, CHR, CHI, IFS, and IFR) and pathogenesis-related genes (PR3 and PR10) in wild-type (R108) at 0, 8, 24 and 48 hours post-inoculation with P. emaculata urediniospores.

FIG. 14: Medicago truncatula irg1 mutants inhibit differentiation of pre-infection structure formation of Phakopsora pachyrhizi.

FIG. 15: A schematic showing the sequence of events for conducting the forward genetic screens to identify M. truncatula genes involved in non-host resistance against P. emaculata.

FIG. 16a-c: Development of pre-infection structures of Puccinia emaculata and Phakopsora pachyrhizi on adaxial and abaxial leaf surfaces. FIG. 16a: pre-infection structure formation of P. emaculata on the adaxial (left panel) and abaxial (right panel) surfaces of wild-type M. truncatula R108 and two independent irg1 mutant alleles (irg1-1 and irg1-2). Urediniospores derived germ-tubes of P. emaculata failed to form appressoria (Ap) on the stomata and penetrate (Pn), therefore, only percentage germination (Ge) and spores with long germ-tubes (Gt) were evaluated as described. FIG. 16b: Pre-infection structure formation of P. pachyrhizi on the adaxial (left panel) and abaxial (right panel) surfaces of wild-type M. truncatula R108 and two independent irg1 mutant alleles (irg1-1 and irg1-2). FIG. 16c: Epifluorescence micrographs showing (arrows) different stages of urediniospores differentiation including long germ-tubes without appressoria (Gt), with appressoria (Ap, arrow) and direct penetration (Pn, arrow) on Medicago. Percentage penetration is calculated by counting the number of dead epidermal cells showing autofluorescence resulting from direct penetration (arrow).

FIG. 17a-b: The irg1 mutant shows partial resistance to Colletotrichum trifolii but not to Phoma medicaginis. FIG. 17a: percentage germination of conidiospores and germ-tubes with no appressoria (Gt) and germ-tubes with differentiated appressoria (Ap) by C. trifolii conidiophores on the adaxial (Ad) and abaxial (Ab) surfaces of wild-type M. truncatula R108 and irg1-1 plants. The fungal structures were stained with lactophenol trypan blue and the percentage of spores forming different pre-infection structures was evaluated, 72 hpi, by counting 20 random fields. The data represents the mean of three independent experiments. FIG. 17b: Symptoms (left panel) and fungal growth evaluated by the GFP-tagged P. medicaginis (right panel) following the inoculation of the spores on the adaxial and abaxial surfaces of wild-type R108 and irg1 mutants of M. truncatula.

FIG. 18a-b: Expression of a Cys(2)His(2) zinc finger transcription factor, PALM1 in irg1 mutant background restores the mutant phenotype. FIG. 18a: Complementation of rust pre-infection structure formation of P. pachyrhizi by expression of MtPALM1 in irg1-1 R108 Tnt1 mutant background (irg1-1::PALM1). P. pachyrhizi spores germinated and formed long germ-tubes within 72 hpi on wild-type R108 and complemented lines (irg1-1::PALM1) but not on the irg1 plants. FIG. 18b: Complementation of rust pre-infection structure formation of P. pachyrhizi by expressing MtPALM1 in irg1-6 M. truncatula A17 deletion mutant.

FIG. 19: Loss of function of IRG1/PALM1 results in loss of epicuticular wax crystal deposition on the abaxial leaf surface of Medicago truncatula. Scanning electron micrographs showing epicuticular wax crystal structure of M. truncatula on the adaxial (left panel) and abaxial (right panel) leaf surfaces of the wild-type R108 and three independent irg1 mutant alleles (irg1-1, irg1-2 and irg1-5) and the PALM1 complemented line (irg1-1::PALM1). Scale bar=5 μm.

FIG. 20: A simplified wax biosynthesis pathway and some CER genes implicated in wax biosynthesis in Arabidopsis (Modified from Kunst and Samuels, 2003; Samuels et al., 2008).

FIG. 21a-c: Wax content and composition of intact leaf (FIG. 21a) and adaxial (FIG. 21b) and abaxial (FIG. 21c) leaf surfaces of wild-type and irg1 mutant alleles (irg1-1 and irg1-2) of M. truncatula. Means±SE of five replications are presented for each data point.

FIG. 22a-d: Composition of adaxial alcohols (FIG. 22a), abaxial alcohols (FIG. 22b), adaxial alkanes (FIG. 22c), and abaxial alkanes (FIG. 22d) of wild-type and irg1 mutant alleles (irg1-1 and irg1-2) of Medicago truncatula. Means±SE of five replications are presented for each data point.

FIG. 23a-b: Effect of epicuticular wax on development of urediniospores of Phakopsora pachyrhizi and Puccinia emaculata. FIG. 23a: Pre-infection structure formation by Phakopsora pachyrhizi urediniospores on uncoated glass slides (Glass-control) and glass slides coated with epicuticular waxes extracted from the abaxial (Ab) or adaxial (Ad) leaf surfaces of wild-type R108 or irg1 plants, 24 hpi. FIG. 23b: Percentage appressorium formation on the detached soybean leaves with native (Wax⁺) or surfaces manipulated to remove the wax (Wax⁻). Urediniospores of P. pachyrhizi (Pp) were spot inoculated on wax⁺ and wax⁻ abaxial leaf surfaces of soybean and the number of appressoria were counted 72 hpi. Similarly, the number of P. emaculata (Pe) appressoria formed on stomata of wax⁺ and wax⁻ abaxial surfaces of switchgrass were counted 72 hpi. Means±SE of three replications are presented for each data point.

FIG. 24a-d: Pre-infection structure formation of Phakopsora pachyrhizi and Puccinia emaculata on the detached leaves with native (Wax⁺) or surfaces manipulated to remove the wax (Wax⁻). FIG. 24a: Symptoms (necrosis resulting from direct penetration) induced by P. pachyrhizi urediniospores inoculated on Wax⁺ and Wax⁻ abaxial leaf surfaces of soybean, 10 dpi. FIG. 24b: Germination of urediniospores of P. emaculata on hydrophilic (uncoated glass) surfaces with long germ-tubes (arrows), 16 hpi. FIGS. 24c-d: P. emaculata (Pe) formed appressoria on stomata of Wax⁺ surfaces FIG. 24c, arrow), but not on the stomata of Wax⁻ surfaces (FIG. 24d, arrow) of switchgrass, 72 hpi.

FIG. 25a-b: Overview of global transcript changes in irg1 mutants. FIG. 25a: Venn diagram representing the overlapping and non-overlapping differentially regulated gene sets among three independent irg1 mutant alleles. FIG.25b: Expression profiles of several genes encoding ECERIFERUM (CER1-4, 6, 8 and 10), PASTICCINO2 (PAS2), β-keto acyl-CoA reductase (KCR1, KCR2), Wax synthase (WSD1) Fatty acyl-ACP thioesterase B (FATB), MYB transcription factor (MYB30) were evaluated using qRT-PCR in leaf samples of wild-type and irg1 mutant alleles (irg1-1, irg1-2 and irg1-5) of Medicago truncatula. Means±SE of three replications are presented for each data point.

### DESCRIPTION OF ILLUSTRATIVE EMBODIMENTS

The studies provided herein surprisingly demonstrate the PALM transcription factor gene, such as the M. truncatula PALM1 plays a primary regulatory role in controlling gene expression involved in leaf development. Plants with mutations in the PALM gene exhibit morphological changes in leaflet structure, such as conversion of trifoliate leaves to pentafoliate leaves. These alterations may allow an increase in biomass such as an increase in amount or ratio of leaf tissues relative to other portions of a plant. Mutant plants also exhibit changes in leaf hydrophobicity and increased resistance to fungal pathogens. For example, spores from Puccinia emaculata and Phakopsora pachyrhizi exhibited greatly reduced growth on plants comprising a disrupted PALM gene. Likewise, these plants were found to be resistant to infectious damage from Colletotrichum trifolii. Further investigation identified a key gene involved in leaf development, SGL1, which is subject to PALM regulation. Thus, disruption of PALM transcription factor expression results in a variety of unexpected characteristics that are agronomically advantageous, including resistance to fungal infection.

One key effect of PALM disruption is enhanced resistance to a variety of fungal pathogens. Accordingly, fungus resistant plant varieties can be produced that comprise a disrupted PALM gene or that express antisense or siRNA sequences that down-regulate PALM expression. For example, plants comprising down-regulated PALM expression exhibit increased resistance to Asian soybean rust (Phakopsora pachyrhizi), thus soybean plants can be produced comprising a down-regulated PALM1 and/or PALM2 gene to provide resistance to rust infection. Such resistance may occur because a germinating or infecting fungal cell may fail to recognize surface chemical (e.g. hydrophobicity) or physical signals (e.g. stomates) at the cuticle surface, and thus may fail to invade mesophyll cells. Likewise, PALM disruption is able to limit P. emaculata and C. trifolii infections and therefore down-regulation of a PALM gene in switchgrass can protect the host plants from rust infection and increase yield for applications such as biofuel production.

Plants comprising down-regulated PALM expression also exhibit increased leafy tissue (an increase in the leaf:stem biomass ratio) which directly impacts forage digestibility. Thus, forage plants down-regulated in PALM expression would be expected to exhibit improved digestibility and nutritional content. Likewise, a major stumbling block to the use of biomass for production fuels is the difficulty in accessing cell wall carbohydrates that store a large portion of the solar energy converted by the plant. An increase in the leaf to stem ratio would therefore result in more accessible carbohydrates for biofuel production and would increase the efficiency and yield of fuel production.

Altered leaf wax amount and content may also be desirable, and no mutants of, for instance, Arabidopsis, Medicago, or maize are known which exhibit a lack of wax crystals on only one side of the leaf. PALM1 mutants also display altered flux of metabolites in biosynthetic pathways leading to cuticular wax production. For instance, an increased flux of precursors into a decarbonylation pathway leading to alkane production and a reduction in accumulation of primary alcohols is noted. Thus, the provided transgenic plants comprise a variety of traits that are useful in the production of agricultural products such as animal feed and biofuel stock that could not previously have been realized.

In certain embodiments, the invention also provides for up-regulation of one or more, or down-regulation of one or more, in a plant of wax biosynthesis-related, P450, and/or pathogenesis-related gene(s) that are differentially regulated in irg1 mutant lines as listed in Tables 5-7, or corresponding ortholog(s) thereof, including CER1, CER2, CER3/WAX2, CER5, CER6, CER8, CER10, KCR1, WSD1, FATB, MYB30, among others. In some embodiments the absolute change (up- or down-regulation) in level of expression of one or more of these genes may be, for instance, 2-fold (e.g. twice the level of expression if up-regulated, or half the level of expression if down-regulated), 4-fold, 5-fold, 7-fold, 10-fold, 13-fold, or more, as well as any intermediate value of change in level of expression.

### I. PLANT TRANSFORMATION CONSTRUCTS

In a certain embodiment DNA constructs for plant transformation are provided. For example, a DNA be an expression for expression of an antisense RNA, siRNA or miRNA that down-regulates expression of a PALM transcription factor. Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the invention, this could be used to introduce genes corresponding to an entire biosynthetic pathway into a plant. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al., (1996).

Particularly useful for transformation are expression cassettes which have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. However, this may not always be the case, and the present invention also encompasses transgenic plants incorporating non-expressed transgenes. Preferred components likely to be included with vectors used in the current invention are as follows.

A. Regulatory Elements

Exemplary promoters for expression of a nucleic acid sequence include plant promoter such as the CaMV 35S promoter (Odell et al., 1985), or others such as CaMV 19S (Lawton et al., 1987), nos (Ebert et al., 1987), Adh (Walker et al., 1987), sucrose synthase (Yang and Russell, 1990), a-tubulin, actin (Wang et al., 1992), cab (Sullivan et al., 1989), PEPCase (Hudspeth and Grula, 1989) or those associated with the R gene complex (Chandler et al., 1989). Tissue specific promoters such as root cell promoters (Conkling et al., 1990) and tissue specific enhancers (Fromm et al., 1986) are also contemplated to be useful, as are inducible promoters such as ABA- and turgor-inducible promoters. The PAL2 promoter may in particular be useful with the invention (U.S. Pat. Appl. Pub. 2004/0049802, the entire disclosure of which is specifically incorporated herein by reference). In one embodiment of the invention, the native promoter of a PALM coding sequence is used.

The DNA sequence between the transcription initiation site and the start of the coding sequence, i.e., the untranslated leader sequence, can also influence gene expression. One may thus wish to employ a particular leader sequence with a transformation construct of the invention. Preferred leader sequences are contemplated to include those which comprise sequences predicted to direct optimum expression of the attached gene, i.e., to include a preferred consensus leader sequence which may increase or maintain mRNA stability and prevent inappropriate initiation of translation. The choice of such sequences will be known to those of skill in the art in light of the present disclosure. Sequences that are derived from genes that are highly expressed in plants will typically be preferred.

It is contemplated that vectors for use in accordance with the present invention may be constructed to include an ocs enhancer element. This element was first identified as a 16 by palindromic enhancer from the octopine synthase (ocs) gene of Agrobacterium (Ellis et al., 1987), and is present in at least 10 other promoters (Bouchez et al., 1989). The use of an enhancer element, such as the ocs element and particularly multiple copies of the element, may act to increase the level of transcription from adjacent promoters when applied in the context of plant transformation.

It is envisioned that PALM coding sequences (or complements thereof) may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters which have higher activity in roots or wounded leaf tissue.

B. Terminators

Transformation constructs prepared in accordance with the invention will typically include a 3′ end DNA sequence that acts as a signal to terminate transcription and allow for the poly-adenylation of the mRNA produced by coding sequences operably linked to a promoter. In one embodiment of the invention, the native terminator of a PALM coding sequence is used. Alternatively, a heterologous 3′ end may enhance the expression of sense or antisense PALM coding sequences. Examples of terminators that are deemed to be useful in this context include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Of course, many examples of suitable marker proteins are known to the art and can be employed in the practice of the invention.

Included within the terms “selectable” or “screenable” markers also are genes which encode a “secretable marker” whose secretion can be detected as a means of identifying or selecting for transformed cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

Many selectable marker coding regions are known and could be used with the present invention including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154, 204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene (Zukowsky et al., 1983) which encodes a catechol dioxygenase that can convert chromogenic catechols; an α-amylase gene (Ikuta et al., 1990); a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene (Ow et al., 1986), which allows for bioluminescence detection; an aequorin gene (Prasher et al., 1985) which may be employed in calcium-sensitive bioluminescence detection; or a gene encoding for green fluorescent protein (Sheen et al., 1995; Haseloff et al., 1997; Reichel et al., 1996; Tian et al., 1997; WO 97/41228). The gene that encodes green fluorescent protein (GFP) is also contemplated as a particularly useful reporter gene (Sheen et al., 1995; Haseloff et al., 1997; Reichel et al., 1996; Tian et al., 1997; WO 97/41228). Expression of green fluorescent protein may be visualized in a cell or plant as fluorescence following illumination by particular wavelengths of light.

### II. ANTISENSE AND RNAi CONSTRUCTS

Antisense and RNAi treatments represent one way of altering agronomic characteristics in accordance with the invention (e.g., by down regulation of a PALM transcription factor). In particular, constructs comprising a PALM coding sequence, including fragments thereof, in antisense orientation, or combinations of sense and antisense orientation, may be used to decrease or effectively eliminate the expression of a PALM transcription factor in a plant and to alter agronomic characteristics (e.g., leaf morphology and disease resistance). Accordingly, this may be used to “knock-out” the function of a PALM transcription factor or homologous sequences thereof.

Techniques for RNAi are well known in the art and are described in, for example, Lehner et al., (2004) and Downward (2004). The technique is based on the fact that double stranded RNA is capable of directing the degradation of messenger RNA with sequence complementary to one or the other strand (Fire et al., 1998). Therefore, by expression of a particular coding sequence in sense and antisense orientation, either as a fragment or longer portion of the corresponding coding sequence, the expression of that coding sequence can be down-regulated.

Antisense, and in some aspects RNAi, methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense and RNAi constructs, or DNA encoding such RNA's, may be employed to inhibit gene transcription or translation or both within a host cell, either in vitro or in vivo, such as within a host plant cell. In certain embodiments of the invention, such an oligonucleotide may comprise any unique portion of a nucleic acid sequence provided herein. In certain embodiments of the invention, such a sequence comprises at least 17, 18, 19, 20, 21, 25, 30, 50, 75 or 100 or more contiguous nucleic acids of the nucleic acid sequence of a PALM transcription factor gene, and/or complements thereof, which may be in sense and/or antisense orientation. By including sequences in both sense and antisense orientation, increased suppression of the corresponding coding sequence may be achieved.

Constructs may be designed that are complementary to all or part of the promoter and other control regions, exons, introns or even exon-intron boundaries of a gene. It is contemplated that the most effective constructs may include regions complementary to intron/exon splice junctions. Thus, it is proposed that a preferred embodiment includes a construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an RNAi or antisense construct which has limited regions of high homology, but also contains a non-homologous region (e.g., ribozyme; see above) could be designed. Methods for selection and design of sequences that generate RNAi are well known in the art (e.g., Reynolds, 2004). These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence. Constructs useful for generating RNAi may also comprise concatemers of sub-sequences that display gene regulating activity.

### III. METHODS FOR GENETIC TRANSFORMATION

Suitable methods for transformation of plant or other cells for use with the current invention are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., 1985), by electroporation (U.S. Pat. No. 5,384,253, specifically incorporated herein by reference in its entirety), by agitation with silicon carbide fibers (Kaeppler et al., 1990; U.S. Pat. No. 5,302,523, specifically incorporated herein by reference in its entirety; and U.S. Pat. No. 5,464,765, specifically incorporated herein by reference in its entirety), by Agrobacterium-mediated transformation (U.S. Pat. No. 5,591,616 and U.S. Pat. No. 5,563,055; both specifically incorporated herein by reference) and by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species, including biofuel crop species, may be stably transformed, and these cells developed into transgenic plants.

A. Agrobacterium-Mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998), alfalfa (Thomas et al., 1990) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

Similarly, Agrobacterium mediated transformation has also proven to be effective in switchgrass. Somleva et al., (2002) describe the creation of approximately 600 transgenic switchgrass plants carrying a bar gene and a uidA gene (beta-glucuronidase) under control of a maize ubiquitin promoter and rice actin promoter respectively. Both genes were expressed in the primary transformants and could be inherited and expressed in subsequent generations. Addition of 50 to 200 M acetosyringone to the inoculation medium increased the frequency of transgenic switchgrass plants recovered.

B. Electroporation

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 9217598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

C. Microprojectile Bombardment

Another method for delivering transforming DNA segments to plant cells in accordance with the invention is microprojectile bombardment (U.S. Pat. No. 5,550,318; U.S. Pat. No. 5,538,880; U.S. Pat. No. 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (PCT Application WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al., 1994), peanut (Singsit et al., 1997), cotton (McCabe and Martine11, 1993), tomato (VanEck et al., 1995), and legumes in general (U.S. Pat. No. 5,563,055, specifically incorporated herein by reference in its entirety).

Richards et al., (2001) describe the creation of transgenic switchgrass plants using particle bombardment. Callus was bombarded with a plasmid carrying a sgfp (green fluorescent protein) gene and a bar (bialaphos and Basta tolerance) gene under control of a rice actin promoter and maize ubiquitin promoter respectively. Plants regenerated from bombarded callus were Basta tolerant and expressed GFP. These primary transformants were then crossed with non-transgenic control plants, and Basta tolerance was observed in progeny plants, demonstrating inheritance of the bar gene.

D. Other Transformation Methods

Transformation of protoplasts can be achieved using methods based on calcium phosphate precipitation, polyethylene glycol treatment, electroporation, and combinations of these treatments (see, e.g., Potrykus et al., 1985; Lorz et al., 1985; Omirulleh et al., 1993; Fromm et al., 1986; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

To transform plant strains that cannot be successfully regenerated from protoplasts, other ways to introduce DNA into intact cells or tissues can be utilized. For example, regeneration of cereals from immature embryos or explants can be effected as described (Vasil, 1989). Also, silicon carbide fiber-mediated transformation may be used with or without protoplasting (Kaeppler, 1990; Kaeppler et al., 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety; (Thompson, 1995) and rice (Nagatani, 1997).

E. Tissue Cultures

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. BACTOAGAR, GELRITE, and GELGRO are specific types of solid support that are suitable for growth of plant cells in tissue culture.

Some cell types will grow and divide either in liquid suspension or on solid media. As disclosed herein, plant cells will grow in suspension or on solid medium, but regeneration of plants from suspension cultures typically requires transfer from liquid to solid media at some point in development. The type and extent of differentiation of cells in culture will be affected not only by the type of media used and by the environment, for example, pH, but also by whether media is solid or liquid.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

Manual selection of recipient cells, e.g., by selecting embryogenic cells from the surface of a Type II callus, is one means that may be used in an attempt to enrich for particular cells prior to culturing (whether cultured on solid media or in suspension).

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the invention will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al., (1975) and MS media (Murashige and Skoog, 1962).

### IV. PRODUCTION AND CHARACTERIZATION OF STABLY TRANSFORMED PLANTS

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the invention. In this case, one would then generally assay the potentially transformed cell population by exposing the cells to a selective agent or agents, or one would screen the cells for the desired marker gene trait.

A. Selection

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphotransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT) which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., 1987). The bar gene has been cloned (Murakami et al., 1986; Thompson et al., 1987) and expressed in transgenic tobacco, tomato, potato (De Block et al., 1987) Brassica (De Block et al., 1989) and maize (U.S. Pat. No. 5,550,318).

Another example of a herbicide which is useful for selection of transformed cell lines in the practice of the invention is the broad spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 describes the isolation of EPSPS mutations which confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103.

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate will typically be preferred, it is proposed that ranges of 0.1-50 mg/l bialaphos or 0.1-50 mM glyphosate will find utility.

An example of a screenable marker trait is the enzyme luciferase. In the presence of the substrate luciferin, cells expressing luciferase emit light which can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells which are expressing luciferase and manipulate those in real time. Another screenable marker which may be used in a similar fashion is the gene coding for green fluorescent protein.

B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 wk, then transferred to media conducive to maturation of embryoids. Cultures are transferred every 2 wk on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soiless plant growth mix, and hardened, e.g., in an environmentally controlled chamber, for example, at about 85% relative humidity, 600 ppm CO₂, and 25-250 microeinsteins m-2 s-1 of light. Plants may be matured in a growth chamber or greenhouse. Plants can be regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 wk on media containing the above ingredients along with 10-5M abscisic acid and then transferred to growth regulator-free medium for germination.

C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and western blots) or by enzymatic function; plant part assays, such as leaf or root assays; and also, by analyzing the phenotype of the whole regenerated plant.

D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this invention may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and 14C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks. Most often changes in response of plants or plant parts to imposed treatments are evaluated under carefully controlled conditions termed bioassays.

### V. BREEDING PLANTS OF THE INVENTION

In addition to direct transformation of a particular plant genotype with a construct prepared according to the current invention, transgenic plants may be made by crossing a plant having a selected DNA of the invention to a second plant lacking the construct. For example, a transgenic event can be introduced into a particular plant variety by crossing, without the need for ever directly transforming a plant of that given variety. Therefore, the current invention not only encompasses a plant directly transformed or regenerated from cells which have been transformed in accordance with the current invention, but also the progeny of such plants.

As used herein the term “progeny” denotes the offspring of any generation of a parent plant prepared in accordance with the instant invention, wherein the progeny comprises a selected DNA construct. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking the desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### VI. PRODUCTION OF FUEL PRODUCTS FROM BIOMASS

The overall process for the production of fuel, such as ethanol from biomass typically involves two steps: saccharification and fermentation. First, saccharification produces fermentable sugars from the cellulose and hemicellulose in the lignocellulosic biomass. Second, those sugars are then fermented to produce ethanol. Thorough, detailed discussion of additional methods and protocols for the production of ethanol from biomass are reviewed in Wyman (1999); Gong et al., (1999); Sun and Cheng, (2002); and Olsson and Hahn-Hagerdal (1996).

A. Pretreatment

Raw biomass is typically pretreated to increase porosity, hydrolyze hemicellulose, remove lignin and reduce cellulose crystallinity, all in order to improve recovery of fermentable sugars from the cellulose polymer. As a preliminary step in pretreatment, the lignocellulosic material may be chipped or ground. The size of the biomass particles after chipping or grinding is typically between 0.2 and 30 mm. After chipping a number of other pretreatment options may be used to further prepare the biomass for saccharification and fermentation, including steam explosion, ammonia fiber explosion, acid hydrolysis.

1. Steam Explosion

Steam explosion is a very common method for pretreatment of lignocellulosic biomass and increases the amount of cellulose available for enzymatic hydrolysis (U.S. Pat. No. 4,461,648). Generally, the material is treated with high-pressure saturated steam and the pressure is rapidly reduced, causing the materials to undergo an explosive decompression. Steam explosion is typically initiated at a temperature of 160-260° C. for several seconds to several minutes at pressures of up to 4.5 to 5 MPa. The biomass is then exposed to atmospheric pressure. The process causes hemicellulose degradation and lignin transformation. Addition of H₂SO₄, SO₂, or CO₂ to the steam explosion reaction can improve subsequent cellulose hydrolysis, decrease production of inhibitory compounds and lead to the more complete removal of hemicellulose (Morjanoff and Gray, 1987).

2. Ammonia Fiber Explosion (AFEX)

In AFEX pretreatment, the biomass is treated with approximately 1-2 kg ammonia per kg dry biomass for approximately 30 minutes at pressures of 1.5 to 2 MPa. (U.S. Pat. No. 4,600, 590; U.S. Pat. No. 5,037,663; Mes-Hartree, et al., 1988). Like steam explosion, the pressure is then rapidly reduced to atmospheric levels, boiling the ammonia and exploding the lignocellulosic material. AFEX pretreatment appears to be especially effective for biomass with a relatively low lignin content, but not for biomass with high lignin content such as newspaper or aspen chips (Sun and Cheng, 2002).

3. Acid Hydrolysis

Concentrated or dilute acids may also be used for pretreatment of lignocellulosic biomass. H₂SO₄ and HCl have been used at high, >70%, concentrations. In addition to pretreatment, concentrated acid may also be used for hydrolysis of cellulose (U.S. Pat. No. 5,972,118). Dilute acids can be used at either high (>160° C.) or low (<160° C.) temperatures, although high temperature is preferred for cellulose hydrolysis (Sun and Cheng, 2002). H₂SO₄ and HCl at concentrations of 0.3 to 2% (w/w) and treatment times ranging from minutes to 2 hours or longer can be used for dilute acid pretreatment.

Other pretreatments include alkaline hydrolysis, oxidative delignification, organosolv process, or biological pretreatment; see Sun and Cheng (2002).

B. Saccharification

After pretreatment, the cellulose in the lignocellulosic biomass may be hydrolyzed with cellulase enzymes. Cellulase catalyzes the breakdown of cellulose to release glucose which can then be fermented into ethanol.

Bacteria and fungi produce cellulases suitable for use in ethanol production (Duff and Murray, 1995). For example, Cellulomonas fimi and Thermomonospora fusca have been extensively studied for cellulase production. Among fungi, members of the Trichoderma genus, and in particular Trichoderma reesi, have been the most extensively studied. Numerous cellulases are available from commercial sources as well. Cellulases are usually actually a mixture of several different specific activities. First, endoglucanases create free chain ends of the cellulose fiber. Exoglucanases remove cellobiose units from the free chain ends and beta-glucosidase hydrolyzes cellobiose to produce free glucose.

Reaction conditions for enzymatic hydrolysis are typically around pH 4.8 at a temperature between 45 and 50° C. with incubations of between 10 and 120 hours. Cellulase loading can vary from around 5 to 35 filter paper units (FPU) of activity per gram of substrate Surfactants like Tween 20, 80, polyoxyethylene glycol or Tween 81 may also be used during enzyme hydrolysis to improve cellulose conversion. Additionally, combinations or mixtures of available cellulases and other enzymes may also lead to increased saccharification.

Aside from enzymatic hydrolysis, cellulose may also be hydrolyzed with weak acids or hydrochloric acid (Lee et al., 1999).

C. Fermentation

Once fermentable sugars have been produced from the lignocellulosic biomass, those sugars may be used to produce ethanol via fermentation. Fermentation processes for producing ethanol from lignocellulosic biomass are extensively reviewed in Olsson and Hahn-Hagerdal (1996). Briefly, for maximum efficiencies, both pentose sugars from the hemicellulose fraction of the lignocellulosic material (e.g., xylose) and hexose sugars from the cellulose fraction (e.g., glucose) should be utilized. Saccharomyces cerevisiae are widely used for fermentation of hexose sugars. Pentose sugars, released from the hemicellulose portion of the biomass, may be fermented using genetically engineered bacteria, including Escherichia coli (U.S. Pat. No. 5,000,000) or Zymomonas mobilis (Zhang et al., 1995). Fermentation with yeast strains is typically optimal around temperatures of 30 to 37° C.

D. Simultaneous Saccharification and Fermentation (SSF)

Cellulase activity is inhibited by its end products, cellobiose and glucose. Consequently, as saccharification proceeds, the build up of those end products increasingly inhibits continued hydrolysis of the cellulose substrate. Thus, the fermentation of sugars as they are produced in the saccharification process leads to improved efficiencies for cellulose utilization (e.g., U.S. Pat. No. 3,990,944). This process is known as simultaneous saccharification and fermentation (SSF), and is an alternative to the above described separate saccharification and fermentation steps. In addition to increased cellulose utilization, SSF also eliminates the need for a separate vessel and processing step. The optimal temperature for SSF is around 38° C., which is a compromise between the optimal temperatures of cellulose hydrolysis and sugar fermentation. SSF reactions can proceed up to 5 to 7 days.

E. Distillation

The final step for production of ethanol is distillation. The fermentation or SSF product is distilled using conventional methods producing ethanol, for instance 95% ethanol.

### VII. DEFINITIONS

Expression: The combination of intracellular processes, including transcription and translation undergone by a coding DNA molecule such as a structural gene to produce a polypeptide.

Forage crops: Crops including grasses and legumes used as fodder or silage for livestock production.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R0 transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

R0 transgenic plant: A plant that has been genetically transformed or has been regenerated from a plant cell or cells that have been genetically transformed.

Regeneration: The process of growing a plant from a plant cell (e.g., plant protoplast, callus or explant).

Selected DNA: A DNA segment which one desires to introduce or has introduced into a plant genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant invention, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Isolation and Characterization of PALM1 Mutants

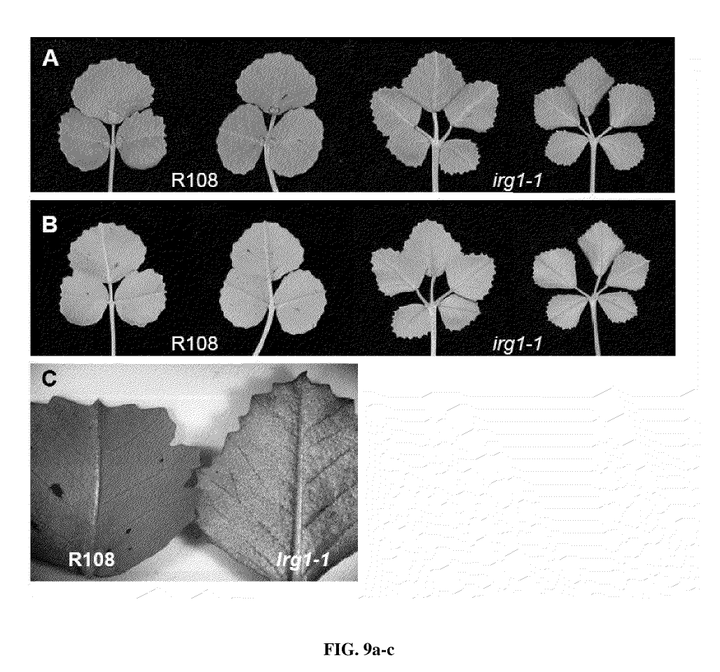

To identify regulators of leaf morphogenesis in legumes, a mutant collection in the model legume M. truncatula derived from fast neutron bombardment deletion mutagenesis was screened. Briefly, seeds of Medicago truncatula cv. Jemalong A17 (wild-type) were exposed to fast neutron radiation at a dosage level of 40 Gy and germinated in a greenhouse with a controlled environment. Approximately 30,000 M2 plants derived from 5,000 M1 lines were screened, resulting in the isolation of two leaf mutants M469 and M534. Mature leaves developed in these two mutants are palmate-like pentafoliate in contrast to the trifoliate wild-type leaves (FIG. 9). These mutants were designated as palmate-like pentafoliatal-1 (palm1-1) and palm1-2. Compared with wild-type compound leaves, which have a terminal and two lateral leaflets, mature leaves in the palm1 mutants have a terminal and two pairs of lateral leaflets clustered at the tip of the petiole. In addition, the two distally oriented lateral leaflets (LLd) are subtended by rachis structures similarly as the terminal leaflet. Scanning electron microscopy and histochemical analysis show the presence of elongated epidermal cells at the surface and three vascular bundles with two on the adaxial side of the rachis structures, indicating changes of LLd to the terminal leaflet (TL) morphology in the palm1 mutant.

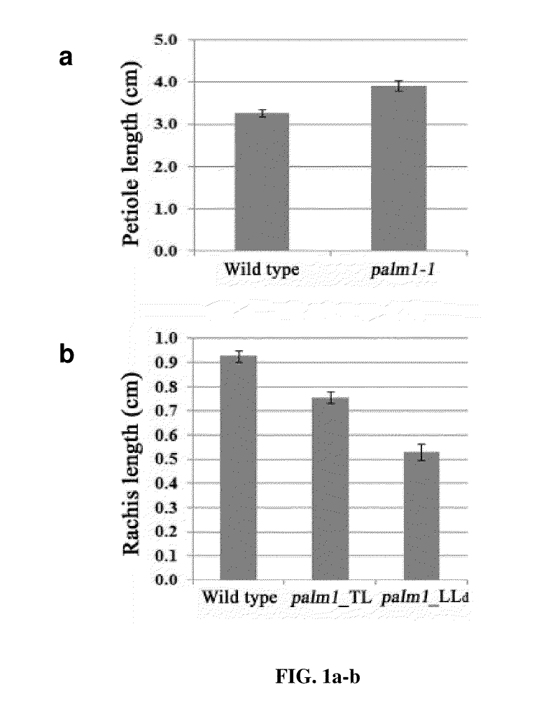

Accompanying these changes, the palm1 mutants also exhibit alterations in the proximal-distal axis of compound leaves. Compared with wild-type leaves, the petiole length of mature leaves in 6-week-old palm1-1 mutant was increased by approximately 20% (FIG. 1A). On the other hand, the length of the central rachis was reduced by approximately 19%, although rachis structures were developed on LLd in the mutant (FIG. 1B).

Scanning electron microscopy was used to identify the earliest morphological alterations during leaf development in the palm1 mutant. Shoot apices of 2- to 4-week-old seedlings were subjected to vacuum infiltration in a fixative solution (5% formaldehyde, 5% acetic acid, 50% ethanol) for 30 min and then kept at room temperature overnight. SEM was carried out as described previously (Wang et al., 2008). In wild-type plants, leaf primordia after initiation (P0 for Plastochron 0) from the periphery of the shoot apical meristem (SAM) developed a pair of stipule (St) primordia at P1, a pair of lateral leaflet (LL) primordia, boundaries between St and LL, and LL and TL at P2, and the differentiation of TL and St as indicated by trichomes developed on their abaxial surface at P3. Leaf development progressed normally in the palm1-1 mutant until the P3 stage, when a pair of extra leaflet primordia, the proximally oriented LL (LLp) developed at the base of LLd, which were initiated at the P2 stage. The earliest morphological alteration in the palm1 mutant, the development of extra leaflet primordia in a basal position, suggest that PALM1 plays a key role in the suppression of the morphogenetic activity in the proximal region of the compound leaf primordium, which is required to maintain the trifoliate morphology of compound leaves and the morphology of LL without the rachis structure in wild-type plants.

### Example 2

### PALM1 Encodes a Cys(2)His(2) Zinc Finger Transcription Factor

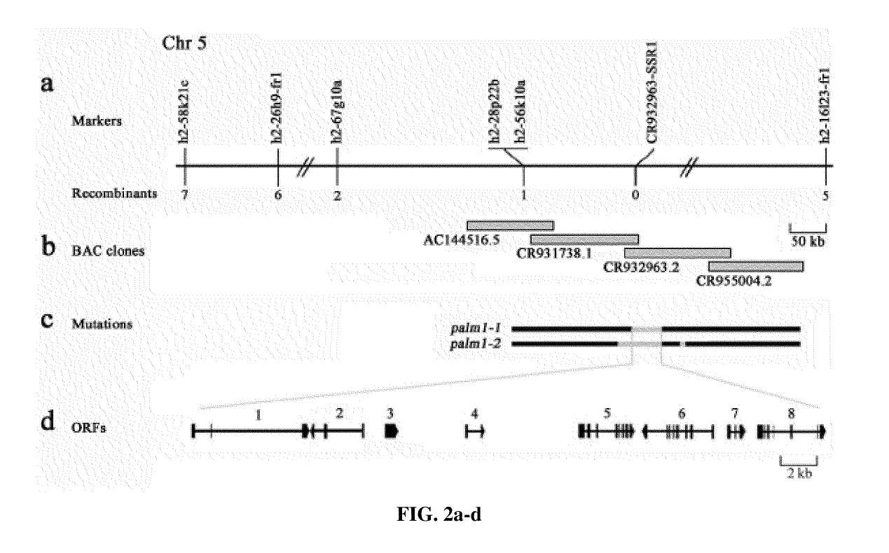

Using a map-based approach, the PALM1 locus was mapped to a 45-kb interval on chromosome 5 (FIG. 2a-c; Table 1). Briefly, F2 mapping populations were derived from crosses between palm1-1 and M. truncatula cv. Jemalong A20. The PALM1 locus was identified using bulked segregant analysis, fine genetic mapping and chromosomal walking. Oligonucleotide primers used in these studies other described below are provided in Table 2.

TABLE 1
Recombination frequency and genetic distance between the
palm1 locus and molecular markers on chromosome 5.

| | Recombination | |
|---|---|---|
| Marker | frequencies ± S.D. | Genetic distance ± S.D. (cM) |
| h2-58k21c | 0.015 ± 0.006 | 1.5 ± 0.006 |
| h2-26h9-fr1 | 0.013 ± 0.005 | 1.3 ± 0.005 |
| h2-67g10a | 0.004 ± 0.003 | 0.44 ± 0.003 |
| h2-56k10a | 0.002 ± 0.002 | 0.22 ± 0.002 |
| h2-28p22b | 0.002 ± 0.002 | 0.22 ± 0.002 |
| CR932963_SSR1 | 0.0 ± 0.0 | 0.0 ± 0.0 |
| h2-16L23-fr1 | 0.011 ± 0.005 | 1.1 ± 0.005 |

TABLE 2
Primer sequences used in studies.

| Name | Forward primer | Reverse primer | Use |
|---|---|---|---|
| CR932963_SSR1 | acgacgttgtaaaacgacCG | TCAAAAACTTTATTTTAGGCATCCA | Genetic |
| | AGCCAATTTTGTTAGACGA | (SEQ ID NO: 2) | mapping |
| | (SEQ ID NO: 1) | | |
| CR931738_2 | GGTTTCTTTGGGATCAAGCA | AAACCGCAGCAAAGAAAAGA | Chromosomal |
| | (SEQ ID NO: 3) | (SEQ ID NO: 4) | walking |
| CR931738_1 | GCACTTGTGTGCAACATTGA | TCGCGTTCATTTAAAACGTG | Chromosomal |
| | (SEQ ID NO: 5) | (SEQ ID NO: 6) | walking |
| Contig77_1 | TGGATGCCACACCCTCTATT | GTTGGGGGTGTCAAATATCG | Chromosomal |
| | (SEQ ID NO: 7) | (SEQ ID NO: 8) | walking |
| Contig77_2 | CCATACAAAGAAGCGGGTGT | AAACTGTTTGGCTCGCTTGT | Chromosomal |
| | (SEQ ID NO: 9) | (SEQ ID NO: 10) | walking |
| Contig77_3 | GTGCTTTCCCCCTCAAAAA | GATAGCTGCTGGATTGGAACA | Chromosomal |
| | (SEQ ID NO: 11) | (SEQ ID NO: 12) | walking |
| Contig77_4 | TGACTTCCCACCTCATCCTC | TACATTCCCCTGGAATTTGG | Chromosomal |
| | (SEQ ID NO: 13) | (SEQ ID NO: 14) | walking |
| Contig77_5 | GTGGCAGTACCCCTGTCTGT | GGTGCAATGGTAAGGTTGCT | Chromosomal |
| | (SEQ ID NO: 15) | (SEQ ID NO: 16) | walking |
| Contig77_6 | ATCAATGACATGGACCCACA | CATCCCTTTGGCTGACCTAA | Chromosomal |
| | (SEQ ID NO: 17) | (SEQ ID NO: 18) | walking |
| Contig77_7 | TGCCCAAATGTGTTTCCATA | AATTTCATGGCTTGGGTTTG | Chromosomal |
| | (SEQ ID NO: 19) | (SEQ ID NO: 20) | walking |
| Contig77_8 | TTGTCTCTCGAATGGTGTGG | CGATCATGCATGGTTTGAAG | Chromosomal |
| | (SEQ ID NO: 21) | (SEQ ID NO: 22) | walking |
| PALM1-gDNA | TCATGAATTCTGCAATATTATTATTATTTAATG | TTAATCTAGAGGCCAGCGTACTTATCTCTTCCTATAC | Comple- |
| | (SEQ ID NO: 23) | (SEQ ID NO: 24) | mentation |
| PALM1ox1 | CCATGGCTACAGATATTGGCCTTC | GGTTACCTCAAGTTGGTGTTGGCTTGTTCC | Ectopic |
| | (SEQ ID NO: 25) | (SEQ ID NO: 26) | expression |
| PALM1ox2 | AAAGGATCCATGGCTACAGATATTGGCC | CCCCTCGAGAGTTGGTGTTGGCTTG | E. coli |
| | (SEQ ID NO: 27) | (SEQ ID NO: 28) | expression |
| PALM1 exp | TTTCTCGAGATGGCTACAGATATTGGCC | TTTCCATGGCTCAAGTTGGTGTTGGCTTG | Local- |
| | (SEQ ID NO: 29) | (SEQ ID NO: 30) | ization |
| PALM1 | AAGTACTCTTTATCATGAATTCTGCAA | GAAGGCACAATCCAGCATTAGC | Promoter |
| promoter | (SEQ ID NO: 31) | (SEQ ID NO: 32) | analysis |
| SGL1 | CCACCTCTCCGTCCCCAA | CAGCGTGCTCACTGTAAAACCA | qRT-PCR |
| | (SEQ ID NO: 33) | (SEQ ID NO: 34) | |
| PALM1 | CCCAACCACCGTTAAATTCTTC | GAAGGCACAATCCAGCATTAGC | qRT-PCR |
| | (SEQ ID NO: 35) | (SEQ ID NO: 36) | |
| MtActin2 | TCAATGTGCCTGCCATGTATGT | ACTCACACCGTCACCAGAATCC | qRT-PCR |
| | (SEQ ID NO: 37) | (SEQ ID NO: 38) | |
| AtEF1a | TGAGCACGCTCTTCTTGCTTTCA | GGTGGTGGCATCCATCTTGTTACA | qRT-PCR |
| | (SEQ ID NO: 39) | (SEQ ID NO: 40) | |
| KNAT1 | TTGGACTGCCAAAAGATTGGA | CCGTGCCGCCGTAATTC | qRT-PCR |
| | (SEQ ID NO: 41) | (SEQ ID NO: 42) | |
| GUS | CCCCAACCCGTGAAATCA | CGCGATCCAGACTGAATGC | qRT-PCR |
| | (SEQ ID NO: 43) | (SEQ ID NO: 44) | |
| PALM1-DNA | CACCATGGCTACAGATATTGGC | TCAAGTTGGTGTTGGCTTGTTC | Genotyping |
| | (SEQ ID NO: 45) | (SEQ ID NO: 46) | |
| KNAT1-DNA | ATGGAAGAATACCAGCATGACA | TTATGGACCGAGACGATAAGG | Genotyping |
| | (SEQ ID NO: 47) | (SEQ ID NO: 48) | |
| BAR | CCGTACCGAGCCGCAGGAAC | CAGATCTCGGTGACGGGCAGGAC | Genotyping* |
| | (SEQ ID NO: 49) | (SEQ ID NO: 50) | |
| Tnt1 | CTCCAGACATTTTTATTTTTCACCAAG | GCATTCAAACTAGAAGACAGTGCTACC | Genotyping* |
| | (SEQ ID NO: 51) | (SEQ ID NO: 52) | |
| SGL1pro-F1 | TATGGTAGCTCATGTGTTGG (SEQ ID NO: 53) | TGAAGAAAGGTAGATGGCAG (SEQ ID NO: 54) | EMSA |
| SGL1pro-F2 | AGGCAATAAGGAAAAGTAGC (SEQ ID NO: 55) | ACCCATAATAATATCCGACC (SEQ ID NO: 56) | EMSA |
| SGL1pro-F3 | AACCACGTCTATCTATAGCC (SEQ ID NO: 57) | TTTGGAAAATTATGAGAAGTGG (SEQ ID NO: 58) | EMSA |
| SGL1pro-F2-1 | AGGCAATAAGGAAAAGTAGC (SEQ ID NO: 59) | CCTCTGATTTGACTTGACTG (SEQ ID NO: 60) | EMSA |
| SGL1pro-F2-2 | AGGCAATAAGGAAAAGTAGC (SEQ ID NO: 61) | AATTGATGCTTTGGGTTGTCG (SEQ ID NO: 62) | EMSA |
| SGL1pro-F2-3 | AGGCAATAAGGAAAAGTAGC (SEQ ID NO: 63) | AGGGTTATTTAGTTCAAATGTTC (SEQ ID NO: 64) | EMSA |
| SGL1pro-F2-4 | AGGCAATAAGGAAAAGTAGC (SEQ ID NO: 65) | TGGTAAGGTCCTGGTCAGTG (SEQ ID NO: 66) | EMSA |
| SGL1pro-F3-1 | AACCACGTCTATCTATAGCC (SEQ ID NO: 67) | TAGTGTTATTCCCAAGACTGG (SEQ ID NO: 68) | EMSA |

*See, e.g., Wang et al., 2008.

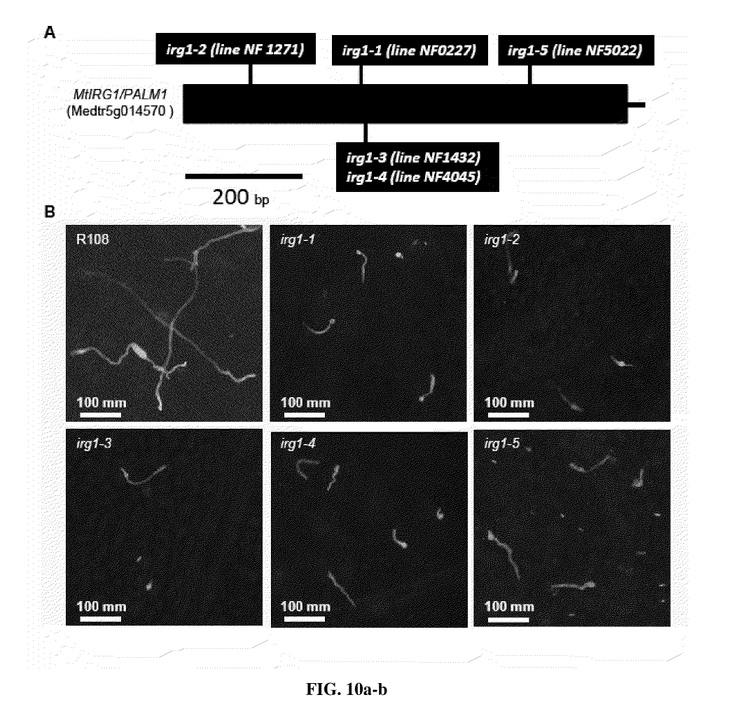

There are eight annotated open reading frames (ORFs) in this genomic interval that are deleted in both palm1-1 and palm1-2 mutants (FIG. 2d; Table 3). Three show syntenic relationships with Arabidopsis thaliana homologues on chromosome 4 (Table 3). To test which ORF is the candidate gene, two other mutant collections were screened and four mutants were isolated with the same phenotype as the original palm1-1 and palm1-2 mutants. These additional mutants were designated palm1-3, -4, -5 and -6 (Table 4). The location of the irg1 mutation in some of these lines is shown FIG. 10. Sequence analysis indicates that palm1-3 carries a 26-bp deletion between positions 243 and 269, and palm1-4, palm1-5 and palm1-6 carry a tobacco Tnt1 retrotransposon at positions 114, 302 and 583, respectively, within the coding region of ORF3 (Table 4). An additional screen for alterations in plant-fungal interactions (i.e. an altered non-host resistance phenotype) as described in Example 6, of Tnt1 insertions in mutant lines derived from R108 was also performed on detached leaves challenged with the switchgrass fungal pathogen Puccinia emaculata. These mutants were termed “irg1” alleles and were found to also localize to PALM1 (IRG1) as shown in Table 4 and in FIG. 10, and as further discussed below.

TABLE 3
Arabidopsis thaliana genes homologous to deleted ORFs in
palm1 mutants.

| | | Homologous | |
|---|---|---|---|
| ORF | Protein | sequence | Annotation |
| ORF1 | 228 a.a. (partial) | At2g35290 | Hypothetical protein |
| ORF2 | 118 a.a. | At2g41580 | Putative non-LTR retroelement |
| | | | reverse transcriptase |
| ORF3 | 251 a.a. | At4g17810 | C2H2 zinc finger domain |
| | | | transcription factor |
| ORF4 | 46 a.a. | No match | Unknown |
| ORF5 | 439 a.a. | At3g47090 | Leucine-repeat receptor- |
| | | | like protein kinase |
| ORF6 | 315 a.a. | At4g17830 | Putative N-acetylornithine |
| | | | deacetylase |
| ORF7 | 128 a.a. | At4g17840 | Hypothetical protein |
| ORF8 | 318 a.a. (partial) | At2g13210 | Putative retroelement polyprotein |

TABLE 4
Mutants and transgenic lines used in these studies.

| | Mutant | | | |
|---|---|---|---|---|
| Line | allele | Gene | Species | Mutation/Construct |
| M469 | palm1-1 | PALM1 | M. truncatula cv. Jemalong | Entire deletion |
| | (irg1-6) | | A17 | |
| M534 | palm1-2 | PALM1 | M. truncatula cv. Jemalong | Entire deletion |
| | | | A17 | |
| GKB483 | palm1-3 | PALM1 | M. truncatula cv. R108 | 26 bp deletion (243-269) |
| NF1271 | palm1-4 | PALM1 | M. truncatula cv. R108 | Tnt1 insertion, 114 bp |
| | (irg1-2) | | | |
| NF0227 | palm1-5 | PALM1 | M. truncatula cv. R108 | Tnt1 insertion, 302 bp |
| | (irg1-1) | | | |
| NF5022 | palm1-6 | PALM1 | M. truncatula cv. R108 | Tnt1 insertion, 583 bp |
| | (irg1-5) | | | |
| NF1432 | Palm1-7 | PALM1 | M. truncatula cv. R108 | Tnt1 insertion |
| | (irg1-3) | | | |
| NF4045 | irg1-4 | PALM1 | M. truncatula cv. R108 | Tnt1 insertion |
| sgl1-1 | sgl1-1 | SGL1 | M. truncatula cv. R108 | Tnt1 insertion, 198 bp* |
| SGL1::GUS | SGL1::GUS | SGL1 | M. truncatula cv. R108 | SGL1::GUS* |
| (Mt) | | promoter | | |
| SGL1::GUS | SGL1::GUS | SGL1 | A. thaliana Co1-0 | SGL1::GUS* |
| (At) | | promoter | | |
| CS3821 | 35S::KNAT1 | KNAT1 | A. thaliana No-0 | 35S::KNAT1** |

*see Wang et al., 2008;
**see, Lincoln et al., 1994.

Secondly, introducing the wild-type ORF3 locus into the palm1-1 mutant rescued the mutant phenotype. For these complementation studies, a genomic fragment, including 2.718-kb 5′-flanking sequence, 0.756-kb ORF and 1.028-kb 3′-downstream sequence of PALM1 was amplified by PCR (see Table 2 for primers) and cloned into pGEM®-T Easy vector (Promega). After sequence verification, the insert was digested with EcoRI and XbaI, and subcloned into pCAMBIA3300. The resulting plasmid was introduced into A. tumefaciens EHA105 and GV3101 strains, and used to transform M. truncatula and A. thaliana, respectively. Based on these results showing a rescue of the palm1-1 mutant phenotype, it was concluded that PALM1 (GenBank accession no. HM038482) corresponds to ORF3, an intron-less gene that encodes a small protein of 251 amino acids. Complementation studies regarding the altered fungal-interaction (irg) phenotype were also performed and are described in Example 10.

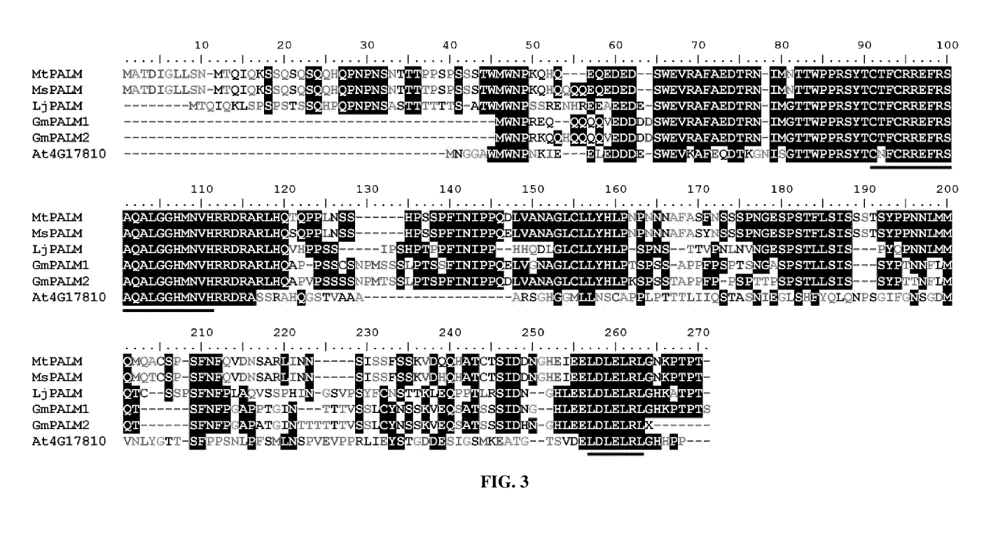

Sequence comparison indicates that PALM1 and its homologues from other plant species share syntenic chromosomal locations and are highly conserved in the EPF-type Cys(2)His(2) zinc finger DNA-binding domain at their N-termini and in the EAR repressor domain identified in the class II ERF transcriptional repressors at their C-termini (Takatsuji, 1999; Ohta et al., 2001) (FIG. 3). Furthermore, PALM1 and its homologues from closely related legume species such as alfalfa (M. sativa; GenBank accession no. HM038483), Lotus japonicus (GenBank accession no. HM038484) and soybean (Glycine max; GenBank accession nos. HM038485 (GmPALM1); HM038486 (GmPALM2)) share a higher degree of sequence similarity than those from more distantly related species such as A. thaliana (FIG. 3).

### Example 3

### Expression Pattern of PALM1 and Subcellular Localization of the Encoded Protein

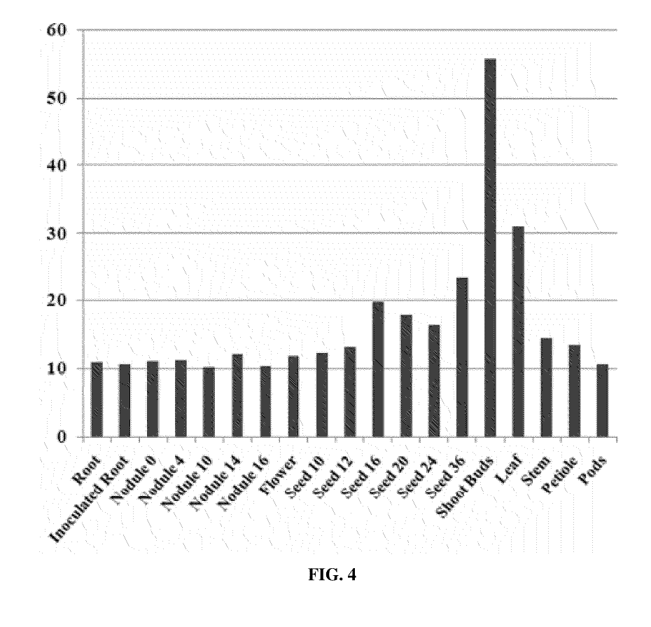

The tissue-specific expression of PALM1 was analyzed by way of in silico expression and RNA in situ hybridization (Benedito et al., 2008). Microarray-based expression analysis indicates that PALM1 transcripts are expressed in vegetative shoot buds, leaves and developing seeds, but remain low or hardly detectable in other tissues including roots, petioles, stems, flowers, pods, and the seed coat (FIG. 4). RNA in situ hybridization was preformed (see, e.g., Coen et al., 1990) using a series of longitudinal sections of vegetative shoot apices shows that PALM1 transcripts were detected in the lateral leaflet primordia as early as the P2 stage. PALM1 transcripts remained low or were barely detected in other tissues including SAM, terminal leaflet and stipules. A sense probe, serving as a negative control, did not give any hybridization signals.

Subcellular localization prediction, using Plant-PLoc (available for instance on the world wide web at: csbio.sjtu.edu.cnlcgi-bin/PlantPLoc.cgi), suggests that PALM1 protein is likely localized to nuclei. To verify this, a green fluorescent protein (GFP)-PALM1 fusion protein transiently expressed using the constitutive Cauliflower Mosaic Virus 35S promoter in onion epidermal cells. Briefly, the plasmid encoding the fusion protein was bombarded into onion epidermal cells using a helium biolistic device (Bio-Rad PDS-1000). The GFP-PALM1 fusion protein was examined using a confocal laser scanning microscope (Leica TCS SP2 AOBS). The fusion protein was specifically localized to nuclei, consistent with its predicted role as a transcription factor.

### Example 4

### PALM1 Negatively Regulates SGL1 Expression

It has been shown that loss-of-function mutations in the M. truncatula FLO/LFY/UNI ortholog SGL1 completely abolished the initiation of LL primordia at the P2 stage, resulting in simple leaves (Wang et al., 2008). SGL1 is expressed in both SAM and entire leaf primordia, the latter of which is partially overlapping with PALM1. However, SGL1 expression is greatly reduced in expanding leaflets (Wang et al., 2008). Thus, SGL1 may be required for the proliferation of LL in the palm1 mutant. Quantitative RT-PCR studies revealed that the SGL1 transcript level was increased by 2.7-fold in vegetative shoot apices in the palm1-1 mutant compared with the wild-type. Briefly, total RNA samples were isolated from tissues using an RNeasy Plant Mini Kit (Qiagen). The quality of the RNA samples was determined by a Nanodrop Analyzer (BioMedical Solution Inc., Stafford, Tex.). Reverse transcription and cDNA synthesis were carried out with 2 μg of total RNA, using an Omniscript RT Kit (Qiagen), oligo(dT)15 columns and oligonucleotide primers described in Table 2.

To further test whether the increase in the SGL1 expression is simply due to an increase in the number of leaflet primordia or an alteration in the expression pattern in the palm1 mutant, the expression of the SGL1pro::uidA (GUS) reporter gene in wild-type was compared to the palm1-1 mutant. In palm1-1 mutant plants the SGL1pro::uidA reporter gene was expressed in all five leaflets, and its expression remained at a high level in expanding leaflets. In contrast, as previously reported, the same reporter gene was only expressed in the SAM and young leaflets, and its expression was greatly reduced in expanding leaflets in the wild-type (Wang et al., 2008). These results indicate that the loss-of-function mutation in PALM1 up-regulated and expanded the spatial-temporal expression of SGL1, a positive regulator of leaflet initiation in M. truncatula (5).

To genetically test the involvement of SGL1 in the proliferation of LL primordia in the palm1 mutant, palm1-3 sgl1-1 double mutants were generated (single mutant alleles all from the R108 ecotype). All leaves that developed in the double mutants were simple, similar to those in the sgl1 single mutant, indicating an epistatic interaction between sgl1 and palm1. The genetic interaction data support the requirement of SGL1 in the proliferation of LL primordia in the loss-of-function palm1 mutant.

To further elucidate potential mechanisms that underlie PALM1 regulation of SGL1 expression, several additional studies were undertaken. The M. truncatula PALM1 gene was ectopically expressed under control of the constitutive 35S promoter in A. thaliana (Col-0) plants and then the transgene (35S::PALM1) was introduced into the plant, through genetic crosses, that carries the SGL1pro::GUS reporter gene (Wang et al., 2008). Ectopic expression of PALM1 did not affect the simple leaf morphology and flower development of the transgenic plants (FIG. 3e, f), but, it almost completely abolished the SGL1pro::GUS gene expression in leaves as indicated by qRT-PCR and GUS staining data, indicating that ectopic expression of PALM1 suppresses the SGL1 promoter activity in A. thaliana leaves.

Next, an electrophoretic mobility shift assay (EMSA) was used to determine the ability of PALM1 to bind the SGL1 5′-flanking sequence. Briefly, 6× His-tagged PALM1 was expressed in E. coli BL21 strain using pET32a vector and purified with the QIAexpressionist™ kit, following the manufacturer's instructions (Qiagen). EMSA was carried out with a Light Shift Chemiluminescent EMSA kit, following the manufacturer's instruction (Pierce). 200 ng of purified recombinant protein and 20 fmol biotin-labeled DNA fragment was used in a 20 μl reaction mix containing 10 mM Tris (pH 7.5), 50 mM KCl, 1 mM DTT, 2.5% glycerol, 0.05% NP-40, 5mM MgCl₂, 0.5 mM EDTA, 5 ng/mL poly(dI•dC) and unlabeled DNA fragment at various molar ratios as competitors. Primer sequences for these studies are listed in Table 2.

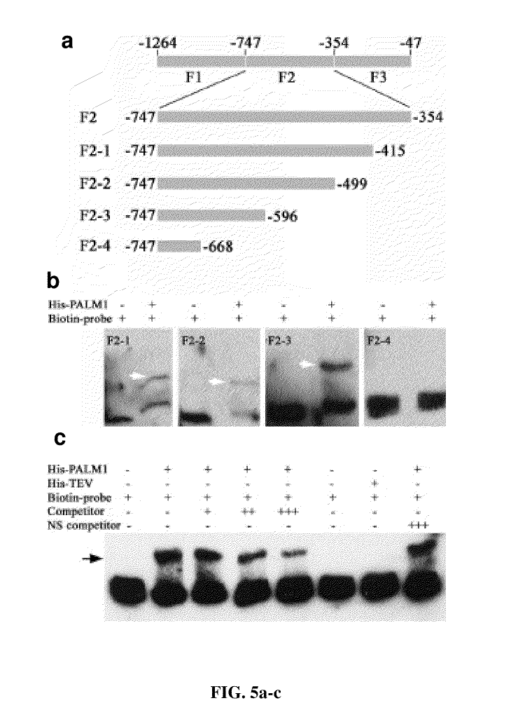

The results of the EMSA studies indicate that PALM1 bound only sequences within the nucleotide region between −354 and −747 upstream of the translation initiation codon. In addition, the interaction was abolished in the presence of high molar ratios of unlabeled specific competitor or the ion chelator EDTA. Furthermore, using a series of deletions, the region in the SGL1 5′-flanking sequence that interacts with PALM1 was narrowed to a 152-bp sequence between nucleotides −596 and −747 (FIG. 5). This interaction is specific, because (i) it was out competed by unlabeled specific sequence, but not by a non-specific sequence from a different region of the promoter; (ii) the interaction was not due to the His tag present in the fusion protein; and (iii) a smaller 80-bp deletion sequence lost the binding activity (FIG. 5c). Collectively, these results indicate that PALM1 may negatively regulate SGL1 expression by directly binding its promoter sequence.

### Example 5

### PALM1 Antagonizes the KNOXI Protein, KNAT1, in A. thaliana

In tomato plants, KNOXI genes are initially down-regulated at the incipient sites of leaf primordia (P0) at the periphery of the SAM and subsequently reactivated in the developing leaf primordia to promote indeterminacy for compound leaf development (Janssen et al., 1998; Parnis et al., 1997; Hareven et al., 1996; Shani et al., 2009). Depending on the developmental context, ectopic expression of TKNs, tomato KNOXI genes, has different effects on leaf shape, supporting a role for TKNs in stage-specific suppression of leaf maturation in tomato (Shani et al., 2009). The KNOXI protein, kn1, has been postulated to play a role in the establishment of the proximal-distal polarity in maize (Zea mays) leaves (Ramirez et al., 2009). In A. thaliana, a plant with simple leaves, ectopic expression of an A. thaliana KNOXI gene, KNAT1/BP, leads to excessive lobing of leaf margins and uneven growth of laminae (Shani et al., 2009; Chuck et al., 1996; Lincoln et al., 1994; Sinha et al., 1993). To test the ability of PALM1 to suppress the effects of over-expression of KNAT1, double transgenic lines were generated, through genetic crossing, that ectopically express both PALM1 and KNAT1 (35S::PALM1 35S::KNAT1). Results showed that both leaf lobing and lamina outgrowth were completely abolished in the double transgenic lines. Quantitative RT-PCR data showed that the KNAT1 transcript level was only slightly reduced in the double transgenic lines compared to the 35S:: KNAT1 lines, in line with the transgene being driven by the constitutive 35S promoter. These results, however, suggest that PALM1 may suppress the effects of over-expression of KNAT1 by regulating its downstream targets, instead of its transcription, in A. thaliana. Although KNOXI proteins are not detected in compound leaves in the IRLC legumes, these results are reminiscent of the previous observation that compound leaf development in IRLC legumes can still respond to ectopic expression of KNOXI genes and suggest that PALM1 is capable of regulating leaf morphogenetic processes that are sensitive to the KNOXI regulation (Champagne et al., 2007).

Mature leaves in M. truncatula, an IRLC legume, are dissected with three leaflets at the tip. Previous studies have shown that the initiation of two lateral leaflet primordia is controlled by the M. truncatula LFY/UNI ortholog SGL1 (Wang et al., 2008). The studies detailed here show that the M. truncatula PALM1 gene encodes a Cys(2)His(2) zinc finger transcription factor that is required to maintain the trifoliate morphology of mature leaves. Several striking phenotypic changes in loss-of-function palm1 mutants, development of two extra leaflets in a basal position, development of the rachis structure on two distally oriented lateral leaflets and alteration of the petiole and rachis length, suggest that PALM1 suppresses the morphogenetic activity in developing leaf primordia and serves as a determinacy factor for leaf morphogenesis in M. truncatula.

The results of the foregoing studies indicate that PALM1 binds a specific sequence in the promoter and negatively regulates the transcription of SGL1. While SGL1 is expressed in the SAM and the entire young leaf primordia, the expression of PALM1 in lateral leaflet primordia partly overlaps with that of SGL1 (Wang et al., 2008). Consistently, the role of PALM1 in the regulation of SGL1 expression and leaf morphogenesis is more pronounced at late stages of leaf development as indicated by the up-regulation and expansion of SGL1pro::GUS reporter gene expression in expanding leaflets in the palm1 mutant compared with wild type and along the proximal-distal axis of leaves as indicated by the altered petiole and rachis length and the ectopic formation of rachis on lateral leaflets in the palm1 mutants (FIG. 1). Thus, the results support a model in which the negative regulator, PALM1, through its own spatial-temporal expression, defines the spatial-temporal expression of SGL1 and the associated morphogenetic activity in leaf primordia and through this regulation determines the trifoliate morphology of mature leaves. In loss-of-function palm1 mutants, the lack of the negative regulation due to loss of PALM1 results in the up-regulation and expansion of SGL1 expression and an increase in the morphogenetic activity, which leads to the development of extra leaflets at a basal position of leaves, ectopic formation of the rachis structure on the distally oriented lateral leaflets and altered development of the proximal-distal axis of leaves.

Taken together, these studies identify PALM1 as a key regulator of dissected leaf morphogenesis in M. truncatula, an IRLC legume. The analysis further shows that PALM1 homologues exist in non-IRLC legumes including soybean and L. japonicus (FIG. 3), in which KNOXI proteins are expressed in leaves and likely associated with compound leaf development in these plants (Champagne et al., 2007).

### Example 6

### Medicago truncatula is an Incompatible/Non Host to ASR and SGR

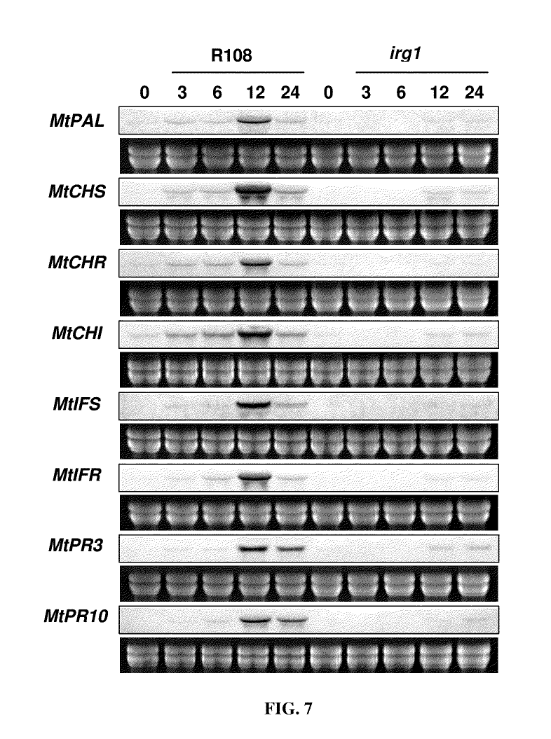

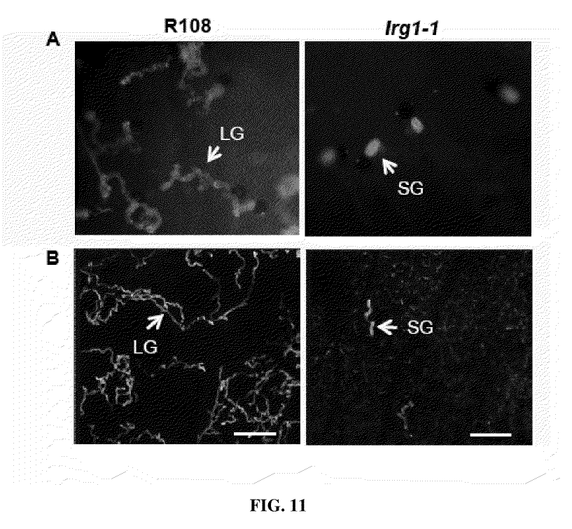

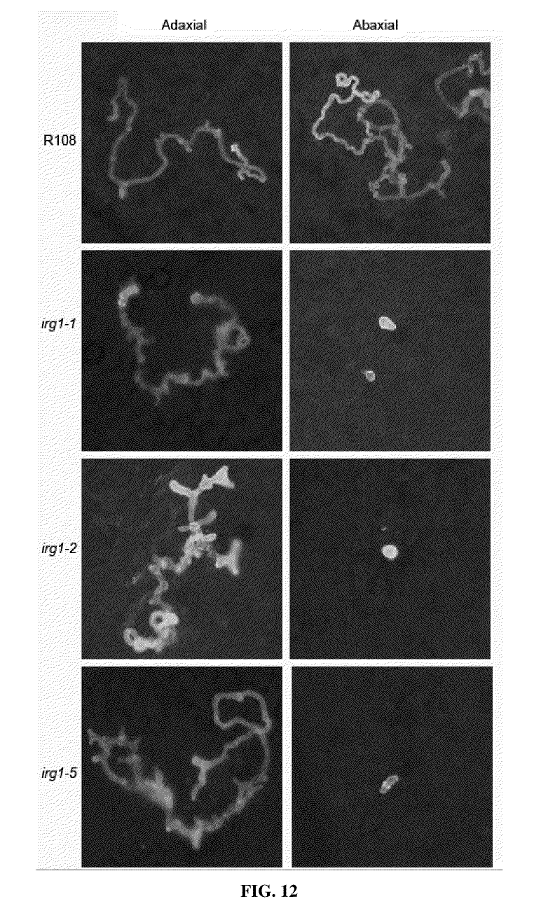

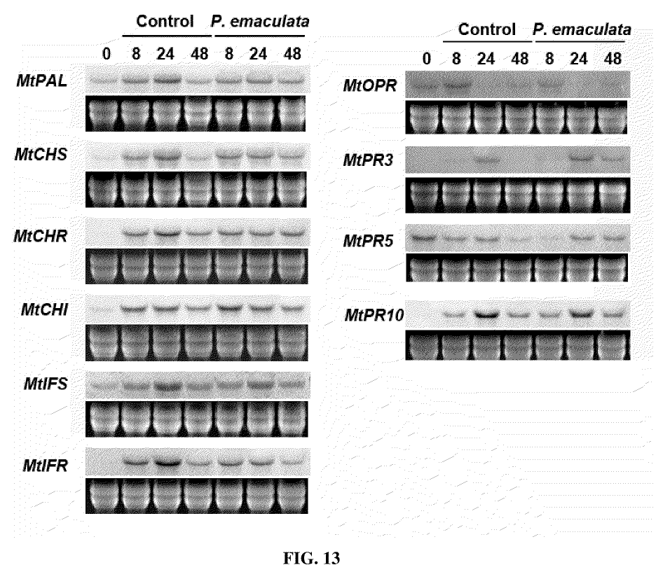

Two economically important fungal infections in plants are Asian soybean rust (ASR) of soybeans caused by Phakopsora pachyrhizi and switchgrass rust (SGR) caused by Puccinia emaculata. However, the studies described here demonstrate that Medicago truncatula, a model legume, displays non-host resistance to P. pachyrhizi and P. emaculata. For instance, when P. emaculata contacts a leaf of M. truncatula, the fungal urediniospores germinate and form long germ-tubes on the leaf surface, but fail to recognize stomata (ingress points) and form appressoria on the stomates (FIG. 11a; FIG. 12). P. emaculata therefore fails to colonize the non-host plant M. truncatula. Pre-infection or pre-haustorial resistance is known to be a common non-host resistance (“NHR”) mechanism to urediniospore-derived parasitic rust fungi, and may also be mediated by activation of defense responses (Heath 1977; Heath 2000). Interestingly, M. truncatula NHR response to P. emaculata was not apparently associated with major transcriptional changes in the phenylpropanoid pathway or pathogenesis related genes (FIG. 7; FIG. 13). In contrast, characterization of the M. truncatula-P. pachyrhizi incompatible interaction has shown that the fungus forms long germ-tubes and directly penetrates M. truncatula epidermal cells resulting in small necrotic lesions. Unlike the non-host interactions of ASR on Arabidopsis (Loehrer et al., 2008), in M. truncatula P. pachyrhizi was able to penetrate and form macroscopic lesions. However, the pathogen failed to sporulate in M. truncatula.

To identify mutants that compromise NHR, 1200 Tnt1 lines representing insertion in approximately 18,000 genes (calculated as per Tadege et al., 2008 where there is an average of 25 insertions per line of which 60% of the insertions are in exons, introns, and UTRs) were screened for loss of NHR to P. emaculata. Detached leaves from about 12 plants of each Tnt1 line were challenged with P. emaculata as described in Example 7.

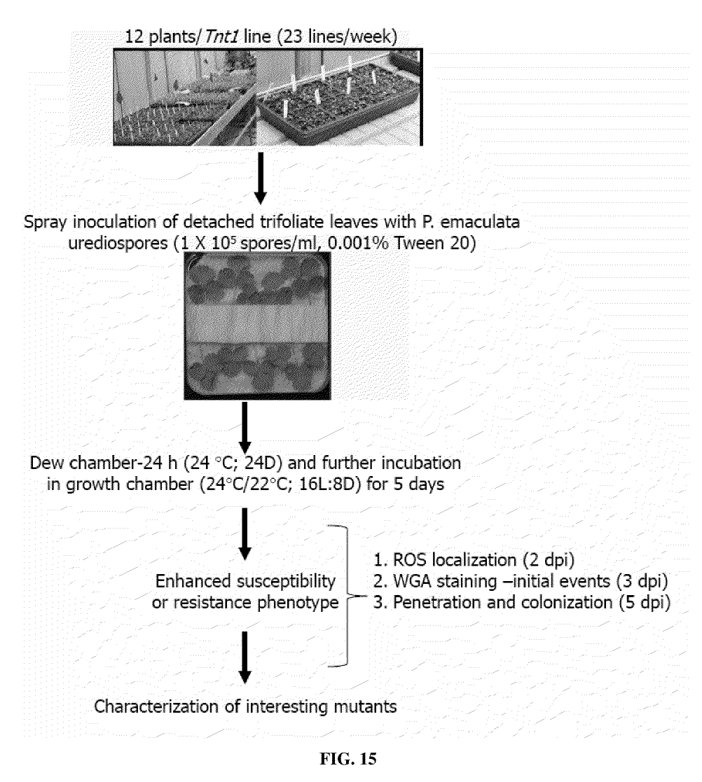

Micro- and macroscopic observations of various steps in the fungal-plant interaction were recorded at 8, 24, and 48 hours post-inoculation (hpi) and five days post-inoculation (dpi) to identify mutants compromised in NHR (FIG. 15).

Initial interactions of P. pachyrhizi or P. emaculata with M. truncatula were recorded by direct observations of inoculated leaves using an Olympus stereo or compound microscope. For fluorescence microscopy, fungal mycelia were stained with wheat germ agglutinin (WGA), coupled to green fluorescent dye Alexa Fluor 488 (WGA-Alexa Fluor® 488; Molecular Probes-Invitrogen; Carlsbad, Calif., USA) as described previously (Uppalapati et al., 2009). Inoculated leaves were stained with 10 μg/mL WGA-Alexa Fluor 488 by a brief vacuum infiltration in PBS followed by 20 min incubation at room temperature. For microscopic observations, after washing with PBS, whole or sections of the leaf were placed on a glass slide and mounted using a cover glass with Dow Corning® (Midland, Mich., USA) high vacuum grease for microscopy. Fluorescence microscopy to document infection process was done using a Olympus epifluorescence microscope or Leica TCS SP2 AOBS Confocal Laser Scanning Microscope (Leica Microsystems Heidelberg GmbH, Mannheim, Germany) equipped with 20× (numerical aperture, 0.70) and 63× (numerical aperture, 1.2) objectives using appropriate laser and excitation filter settings (WGA-Alexa Flour 488-488 nm). Chloroplast autofluorescence was captured by exciting with the 647 nm line of the Argon-Krypton laser and emission detected at 680 nm. Series of optical sections (z series) were acquired by scanning multiple sections and the z-series projections were done with the software provided with the Leica TCS SP2 AOBS CLSM.

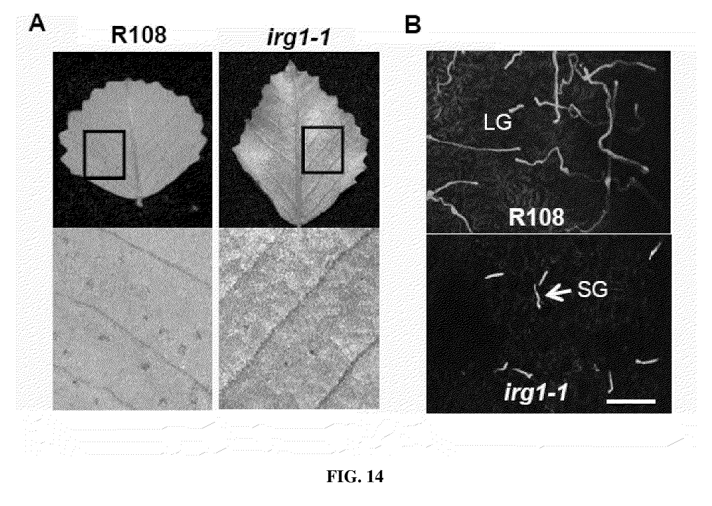

These results (e.g. FIG. 10b; FIG. 12; FIG. 14) suggested that M. truncatula may be employing a novel resistance mechanism to contain these non-host (non-adapted) pathogens. Thus, M. truncatula mutants obtained upon screening for enhanced susceptibility or resistance to ASR penetration could lead to the identification of novel genes that could be utilized for genetic improvement of soybean and switchgrass, respectively.

### Example 7

### Forward Genetic Screening of the M. truncatula Tnt1 Populations

To identify M. truncatula genes that confer resistance to ASR and/or SGR a forward genetic screen using Tnt1 mutant populations employed (see, e.g., Tadege et al., 2008). A maximum of twelve plants per each Tnt1 lines were challenged with ASR or SGR as follows:

Seeds of Medicago truncatula cv. R108, Jemalong A17 or Tnt1 lines were scarified for 8 min using concentrated sulfuric acid, washed thrice with distilled water and germinated on moist filter papers. Two days after germination in darkness at 24° C., 12 seedlings per each Tnt1 line were transferred to soil (one seedling per cell in 6×12 celled trays). Following three week incubation in green house the plants were transferred to growth chambers located in a USDA-APHIS approved facility to conduct soybean rust or switchgrass rust inoculation assays, each described below.

An Illinois isolate of the ASR pathogen P. pachyrhizi was obtained from Dr Glen L Hartman, National Soybean Research Center, Urbana, Ill., and was maintained on the susceptible soybean cultivar (Glycine max cv. Williams) maintained in a growth chamber at 22° C./19° C. with 12L:12D. Fresh urediniospores were collected using a gelatin capsule spore collector designed by the CDL (St. Paul, Minn.) and suspended in distilled water with 0.01% Tween 20. 15 ml of the spore suspension adjusted to 1×10⁶ spores/ml is used to spry inoculate one try (as described above) of four-week old Tnt1 plants using an artist air-brush (Paasche Airbrush Co., Chicago, Ill., USA) set at 2 PSI with a portable air-pump (Gast Mfd. Co., Benton Harbor, Mich. USA) for uniform spore deposition. The inoculated plants were maintained in a dew chamber for 24 h with 100% humidity maintained at 19° C.; 24D. The plants were then transferred to growth chamber (22° C./19° C. with 12L:12D) and incubated further to allow the symptom development.

The SGR isolate was collected from Oklahoma (PE-OK1, Uppalapati et al., unpublished) and was maintained on a susceptible low-land switchgrass (Panicum virgatum L.) Summer genotype. Spores were collected and prepared as described for soybean rust above were used to inoculate detached leaves or whole plants grown in trays. One trifoliate leaf collected from each of the Tnt1 line (maximum of 12 seedlings/Tnt1 line) was spot inoculated with 1×10⁵ spores/ml (0.01% Tween 20) and incubated at 24° C. with 16L:8D. The detached leaves were maintained on moist filter papers or floated on water in 6 well plates.

To date, 1,200 Tnt1 lines have been screened. Considering the fact that there are on average 15-25 insertions per line, approximately 20,000 genes have been screened for their involvement in soybean and switchgrass rust resistance. One of the identified mutants which a displayed rust resistance phenotype termed inhibitor of rust germ-tube differentiation (“irg1”) was further characterized.

### Example 8

### irg1 Mutant Displays Resistance to ASR and SGR and RER Encodes the PALM1 Zinc Finger Transcription Factor

During the forward screen, a Tnt1 mutant line was identified that showed dramatic differences in the initial interactions with P. emaculata. Interestingly, the spores of P. emaculata germinated, but failed to undergo further differentiation and growth on an irg1 mutant. Although the spores germinated, they formed very short-germ tubes on irg1 mutant when compared to the long-germ tubes on the wild-type M. truncatula leaves. These results suggested that the irg1 mutation results in alterations in surface signal required for initial host-pathogen interactions.

To further test if the irg1 mutant displays similar responses to a direct penetrating rust fungus, M. truncatula was inoculated with P. pachyrhizi spores and the initial stages of interaction were recorded. Briefly, approximately 100 spores in 10 μl aliquots were placed on adaxial or abaxial surface of the detached leaves from four-week old M. truncatula wild-type (R108) or irg1 mutant leaves and incubated in the dark overnight, before transfer to a growth chamber (22° C./19° C. with 12 hours-light/12 hrs-dark cycle). For early stages of pre-infection adhesion assays, 1-24 hours post-inoculation (hpi) the leaves were washed 2-3 times in a Petri-dish with water (0.05% Tween 20) to remove free floating spores and leaves were stained by floating in a petri dish with PBS solution containing 10 μg/ml WGA-Alexa Fluor 488 to visualize the fungal germ tubes and appressoria. The number of spores following washing were evaluated microscopically. The number of spores remaining attached at 1 hpi was compared to the initial number of spores before washing, and was used to calculate spore adhesion percentage. The number of spores forming germ-tubes and/or appressoria was evaluated 24 hpi from 20 random fields on three independent leaves. The average was used to calculate percentage germination, and the number of spores with and without appressoria. Penetration percentage of P. pachyrhizi spores was obtained by the counting the number of dead epidermal cells resulting from direct penetration by P. pachyrhizi (auto fluorescence) at 72 hpi from 20 random fields per each inoculation site, and was used to calculate the percentage of penetration.

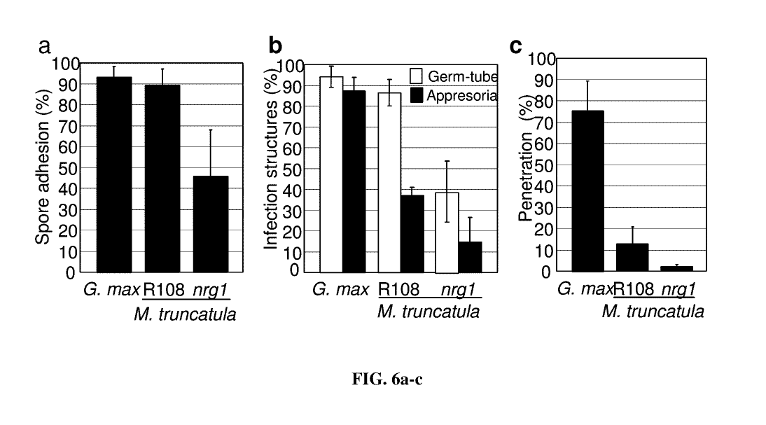

On M. truncatula, urediniospores of P. emaculata germinated and formed long germ-tubes, but failed to form appressoria on the stomates, thus failing to penetrate the leaves. Therefore only the number of germinated spores at 24 hpi, and the number of spores that formed long germ-tubes without appressoria, 48 hpi, were evaluated as described for the P. pachyrhizi-inoculated leaves. Surprisingly, ASR inoculated irg1 leaves showed less necrotic symptoms when compared to the R108. Microscopic observations, showed that on the host (soybean), ASR spores adhere, germinate and form short germ-tubes with appressoria (penetration structures) and directly penetrate the epidermal cells. On wild-type M. truncatula, they adhere at high percentage as on the host plant, but form long germ-tubes with low frequency of appressoria and penetration (FIG. 6a-c). However, in the irg1 mutant the ability of the spores to adhere and form long germ-tubes with appressorial formation was severely compromised, resulting in decreased penetration and necrotic symptoms (FIG. 6a-c). Consistent with failed penetration, the irg1 mutant showed no expression of penetration or pathogenesis-related gene responses (FIG. 7).

Interestingly, the irg1 mutant showed a five-leaf phenotype. It was also demonstrated that the leaves of the irg1 mutant display less hydrophobicity possibly indicating alterations in leaf wax or surface structures. It was confirmed that IRG1 (Tnt1 line, NF0227) encodes PALM1 the same Cys(2)His(2) zinc finger transcription factor that controls trifoliate leaf development in M. truncatula characterized in examples 1-5. By evaluating several alleles including three previously identified Tnt1 lines (NF1271 with the palm1-4 allele, also termed irg1-2; NF0227 with the palm1-5 allele also termed irg1-1; NF5022 with the palm1-6 allele also termed irg1-5); a A17 deletion mutant, A17 palm1-1; and NF1432 with the palm1-7 allele also termed irg1-3) (see also FIG. 10), it was also confirmed that the irg1 phenotype results from loss of PALM1. Thus, the results suggested that loss-of-function mutants of PALM1 display an additional irg phenotype apparently resulting from pathway interactions leading to altered surface signaling in pathogenesis.

### Example 9

### irg1 Mutants Show Partial Resistance to Colletotrichum trifolii but not to Phoma medicaginis and Sclerotinia sclerotiorum

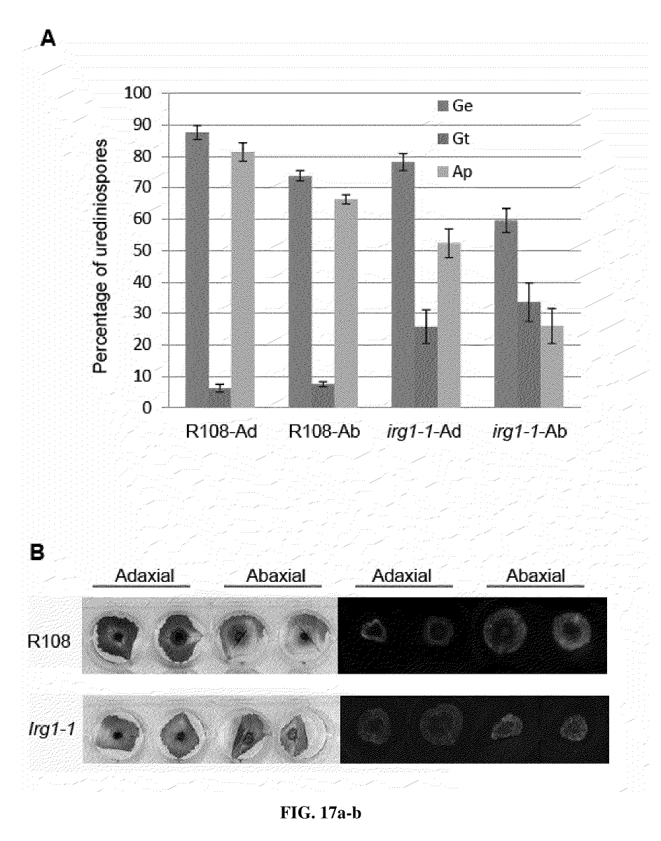

To test if irg1 (palm1) lines exhibit broad-spectrum tolerance/resistance, wild-type (R108) and PALM1 Tnt1 insertion lines were challenged with several other fungal pathogens of alfalfa as follows: For infection assays with Phoma medicaginis, a necrotrophic pathogen (FIG. 17b), P. medicaginis P-GFP inoculum were maintained on potato-dextrose agar (PDA; Becton, Dockinson & Co., Sparks, Md.) with hygromycin (100 μg/ml). To promote conidal formation the cultures were grown on YPS agar (0.1% each, yeast extract, peptone, glucose and 1.5% agar) with hygromycin (100 μg/ml) for 2 weeks and condia were harvested with water. Triofoliate leaves from six week-old clonally propagated wild-type and transgenic antisense alfalfa lines were harvested and spot inoculated with 10 μl of suspension containing 1×10⁶ spores/ml in 0.05% Tween 20 on adaxial and abaxial surface. The mock (distilled water, 0.05% Tween 20) and fungal inoculated leaves were floated on water, sealed and incubated at 22° C./19° C., 16-h photoperiod, photon flux density 150-200 μmol m-2 sec-1). Disease development was monitored every day until 10 days post inoculation. The screening test was repeated twice. Twelve independent leaves were used in each experiment. GFP-tagged fungus and autofluorescence of the chloroplast were visualized using a stereomicroscope (Olympus, SZX16) equipped with epiflourescence.

For infection assays with Sclerotinia sclerotiorum agar plugs (5 mm, dia.) from growing regions of S. sclerotiorum isolate were obtained, grown on PDA media and used as inoculum. Leaves from four week-old wild-type and irg1 mutant lines were inoculated with one agar plug per leaf and fungal inoculated leaves were placed on moist filter papers, sealed and incubated at 22° C./19° C., 16-h photoperiod, photon flux density 150-200 μmol m-2 sec-1). Two days after inoculation with S. sclerotiorum, the size of necrotic region was checked and disease severity assessed based on percentage leaf area infected. The inoculation assay with S. sclerotiorum was repeated 6 times.

For infection assays with Colletotrichum trifolii, a hemibiotrophic pathogen that forms pre-infection structures (FIG. 17a), C. trifolii race 1 was maintained on PDA media. Conidia from 10-14 days old cultures were harvested in water washed and re-suspended in sterile distilled water. Leaves from four week-old wild-type and irg1 mutant lines were harvested and spot inoculated with 10 μl of suspension or spray inoculated with a suspension containing 1×10⁶ spores/ml in 0.005% Tween 20. The fungal structures were stained with lactophenol trypan blue and the percentages of spores forming different pre-infection structures was evaluated 72 hpi, by counting 20 random fields. Three independent experiments were performed.

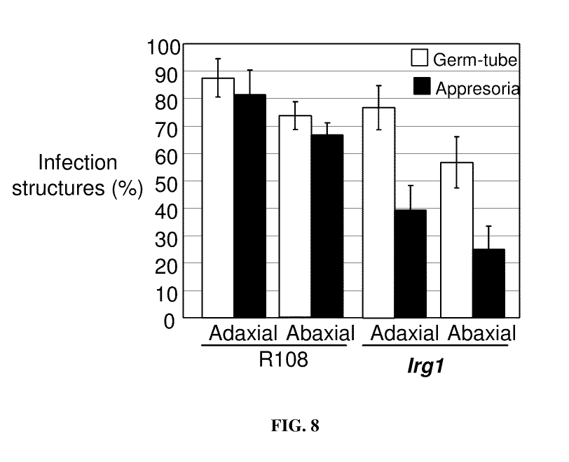

Results of the inoculation studies demonstrated that five days post-inoculation with C. trifolii, the leaves of wild-type showed severe anthracnose disease symptoms and formation of fruiting bodies when compared to the irg1 lines that supported less formation of infection structures (germ-tubes and appressoria) when compared to the wild-type (FIG. 8). The percentage of C. trifolii spore germination and formation of pre-infection structures (appressoria) was impaired on the abaxial leaf surface of irg1-1 plants compared to wild type R108 (FIG. 17a). Percentages of germination and appressoria formation were also slightly reduced (by ˜10%) on the adaxial surface of irg1-1 mutants when compared to wild-type. However, an irg1 mutant did not show significant tolerance to P. medicaginis as measured by symptom development or in planta fungal growth on either abaxial or adaxial leaf surfaces (FIG. 17b). These results suggest that irg1 mutants impact those fungi that form pre-infection structures in response to surface signals. The irg1 mutation may thus result in resistance to a broad-spectrum of fungi that form penetration structures (such as appressoria) in response to surface (thigmo or chemo) signals.

### Example 10

### Differential Effects on Fungal Spore Germination and Penetration on Abaxial Surfaces of Leaves of the irg1 Mutant

The abaxial (underside of leaf, facing away from stem) but not adaxial (top of leaf, facing toward stem) leaf surfaces of irg1-1 plants were glossy in appearance when compared with wild-type M. truncatula R108 plants (FIG. 14a; FIG. 9), in which neither surface is glossy. This suggested possible alterations in epicuticular wax formation on the abaxial leaf surface. It is known that for some host-specific biotrophic pathogens including Erysiphe pisi and Blumeria graminis, the components of abaxial wax may specifically promote pre-infection structures (e.g. Gniwotta et al., 2005; Hansjakob et al., 2010). Thus, two different inoculated nonhost rust pathogens with different styles of pre-infection processes were further studied on irg1-1 plants to determine if surface cues for adherence or germ-tube differentiation might alter the plant-pathogen interaction.

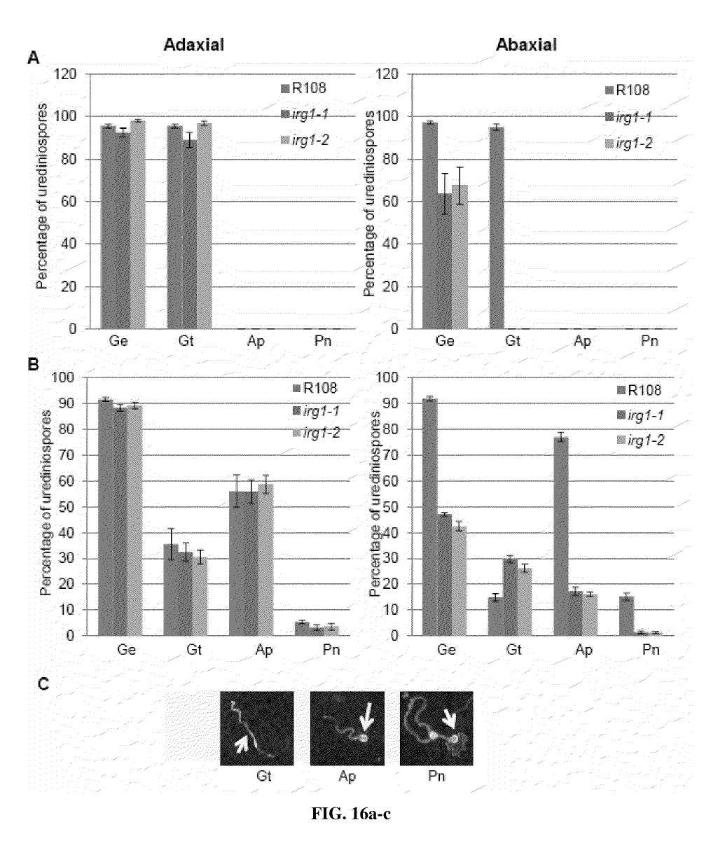

Pre-infection structure formation by P. emaculata and C. trifolii on abaxial leaf surfaces of M. truncatula was examined (FIG. 16; see also Example 11). On the adaxial leaf surfaces of both wild-type and irg1 plants, almost 90% of inoculated urediniospores of P. emaculata that germinated were able to form long germ-tubes with no appressoria on stomata. Similarly, no inhibition of urediniospore germination or germ-tube elongation was observed on the abaxial leaf surface of wild-type plants (FIG. 16a). However, only about 60% of spores germinated on the abaxial leaf surface of the irg1 mutant plants, and almost one half of germinated spores failed to undergo any further differentiation (FIG. 16a).

Unlike P. emaculata, P. pachyrhizi is a broad host range and direct-penetrating biotrophic rust fungus. It has been suggested that hydrophobic or chemical signals are not required for pre-infection structure formation by P. pachyrhizi (Koch and Hoppe, 1988; Goellner et al., 2010). In vivo assays conducted on adaxial surfaces showed no significant differences in percentage germination, appressorium formation, or epidermal penetration by P. pachyrhizi between wild-type M. truncatula R108 and three independent mutant lines with distinct irg1 alleles (FIG. 16b). P. pachyrhizi had a slightly higher percentage of appressorium formation and penetration rate when inoculated on the abaxial surface of wild-type (R108) M. truncatula, as compared to the adaxial surface (FIG. 16b). However, on the abaxial surface of irg1-1 mutants, about 50% of spores failed to germinate (e.g. FIG. 16b, “abaxial”). Strikingly, only 20% of the spores formed appressoria on the abaxial side of leaves of irg1 mutants, while ˜75% of spores formed appressoria on the abaxial surface of R108 wild-type leaves (FIG. 16b). These results demonstrate that the abaxial surface of irg1 may either inhibit or lack surface cues required for differentiation of pre-infection structures formation by P. pachyrhizi.

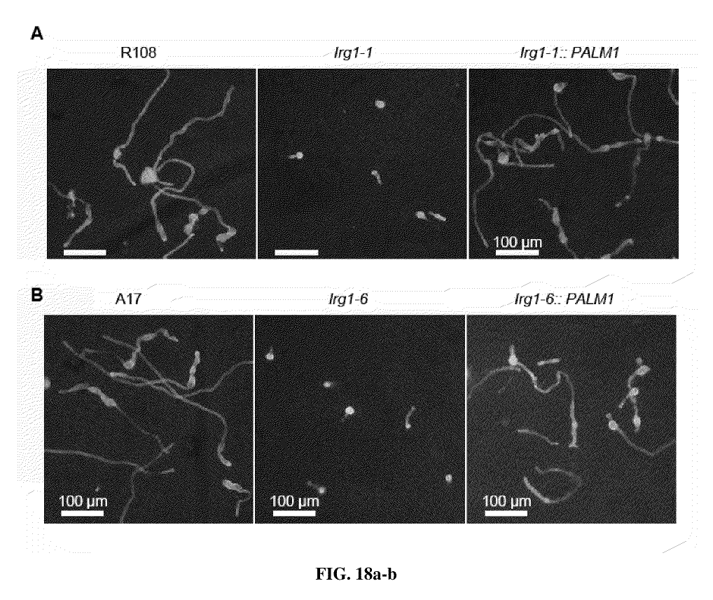

The association of the irg1 phenotype with PALM1 was further confirmed by evaluating several irg1 alleles identified from Tnt1 lines in addition to those found to have an insertion in PALM1 (e.g. FIG. 10). Along with the irg lines, in some of the screens for altered interactions between a fungal pathogen and the non-host M. truncatula, Tnt1 insertion line NF5022 (comprising the palm 1-6 allele) and the M. truncatula ecotype A17 deletion line M469 (comprising palm1-1; Chen et al., 2010) were also tested for their IRG phenotype. The formation of pre-infection structures of P. pachyrhizi and P. emaculata was severely impaired on all identified alleles of palm1 (e.g. see FIGS. 10, 12), indicating that the loss of function mutation of PALM1 is responsible for the irg1 phenotype. Thus, palm1-6 and palm1-1 were also designated as irg1-5 and irg1-6, respectively. To further confirm that the irg1 phenotype results from a loss of function in PALM1, the mutant phenotype of both irg1-1 and irg1-6 expressing PALM1 under its native promoter was complemented (see FIG. 18). The complemented lines did not show any inhibition of formation of rust pre-infection structures. These results show that the function of a gene involved in controlling leaf morphology also plays a role in determining non-host fungal resistance.

### Example 11

### Irg1 Displays an Epicuticular Wax Mutant Phenotype

Based on fungal spore germination and development results, possible alterations were examined for abaxial surface chemical and/or physical signals present in irg1 mutants. Scanning electron microscopy (SEM) analyses were performed as described previously (Zhang et al., 2005). Briefly, leaves from the top two internodes from the major stem were harvested and air-dried at room temperature in a Petri-dish. Air-dried leaves were mounted on stubs and coated with approximately 20 nm of 60/40 gold-palladium particles using a Hummer VI sputtering system (Anatech Ltd., Springfield, Va., USA). Coated surfaces were viewed using a JEOL JSM-840 scanning electron microscope at 15 kV (JEOL, Peabody, Mass., USA).

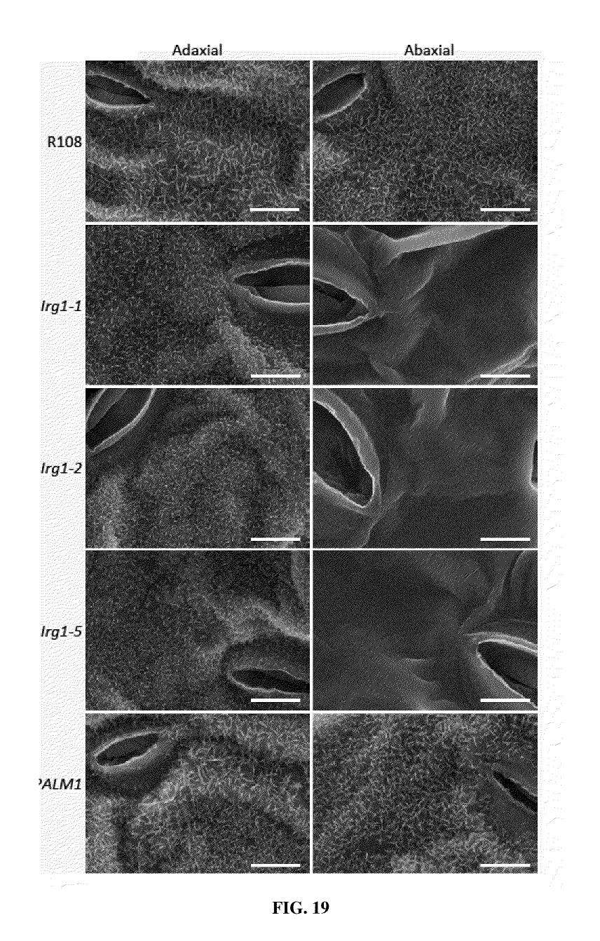

The SEM analyses of air-dried leaf samples showed no major differences in density or physical structure of epicuticular waxes between adaxial and abaxial leaf surfaces of wild-type M. truncatula plants (FIG. 19). However, plants comprising irg1-1 or other irg alleles completely lack epicuticular wax crystals on the abaxial leaf surface, but not the adaxial surface (FIG. 19). Leaves from the transgenic complemented line irg1-1::PALM1) did not display significant differences in wax crystal deposits between adaxial and abaxial leaf surfaces, and were similar in this regard to wild-type leaves. This is the first report known to the inventors of a mutant with a defect in abaxial epicuticular wax deposition due to a mutation in a gene also involved in leaf morphogenesis.

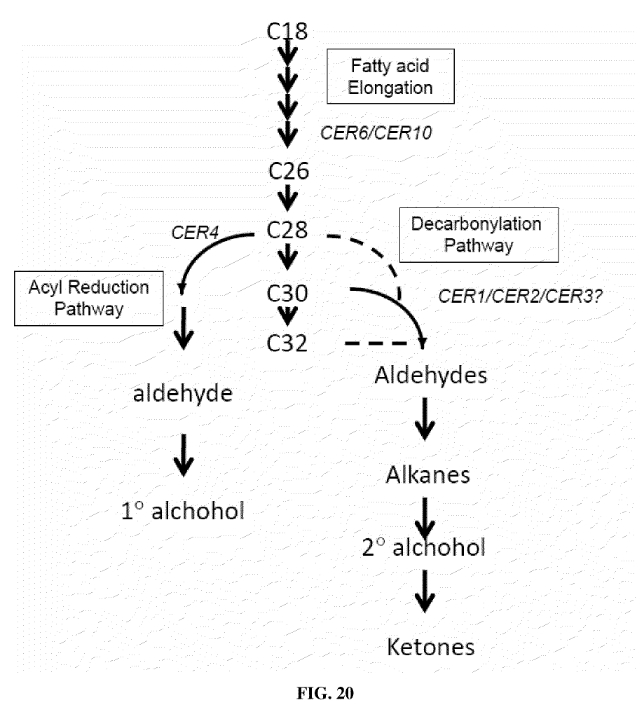

To further understand the nature of compositional changes in epicuticular waxes of irg1 plants, cuticular waxes were extracted from wild-type R108, irg1-1, and irg1-2 lines. A chemical pathway for acyl-reduction and decarbonylation pathways in leaf wax biosynthesis is shown in (FIG. 20).

Total leaf cuticular wax extraction and analysis was conducted as described previously (Zhang et al., 2005). Briefly, leaf samples were collected from one leaflet of each of the top two expanded trifoliates/pentafoliates excised from major stems of well watered wild type R108 and irg1 mutant lines. The two leaflets were combined as one sample. Each sample was added to 10 mL of GC-MS grade hexane (Sigma-Aldrich, St. Louis, Mo., USA). Tissues were agitated for 2 minutes and the solvent was decanted into new glass tubes. The same amount of hexane was added to rinse the tissues and tubes for 10 seconds, and was pooled into the sample tube. Hexane was evaporated to approximately 1 mL under a nitrogen stream, and the sample was transferred to a 2 mL autosampler vial and then evaporated completely. Dried extracts were derivatized using N-methyl-N-trimethylsilyltrifluoroacetamide (MSTFA+1% TMCS; Pierce Biotechnology, Rockford, Ill., USA) and were run on an Agilent 7890A gas chromatograph (Agilent, Palo alto, Calif., USA) using a splitless injection as described (Zhang et al. 2005). Quantification was done using the area of the ions with m/z M-15, 117, 57, and 218 for fatty alcohols, fatty acids, alkanes, and sterols, respectively. The amount of total wax as well as each cuticular was constituent was expressed per unit of leaf area. Leaf areas were determined using a leaf area meter (LI-3000; Li-Cor, Lincoln, Nebr., USA). All values represent averages of five replicates ±SE.

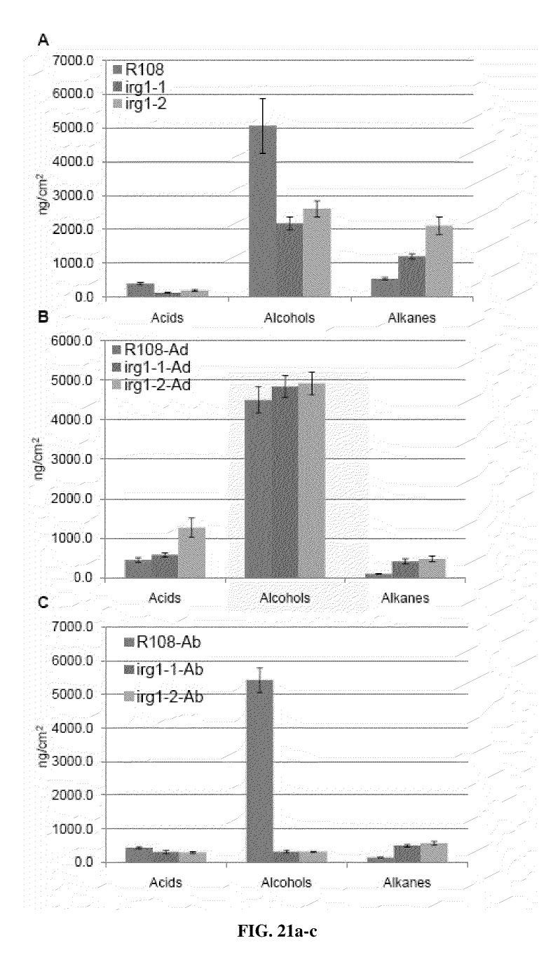

Since SEM pictures showed a lack of epicuticular wax crystals on the abaxial surface of leaves of irg1 mutant lines and inhibition of rust spore differentiation was observed only on the abaxial surfaces, it was hypothesized that the abaxial surface of irg1 plants may either lack a particular wax constituent that promotes germ-tube differentiation, or may accumulate one or more inhibitory constituents of waxes. Total epicuticular waxes isolated from intact leaves were ˜1.7 fold higher per leaf area in R108 compared to irg1 mutants. The amount of total acids and alcohols in irg1-1 leaves were about 3.2 and 2.3- fold lower, respectively, than in R108 leaves (FIG. 21). However, total alkanes were 2.2-fold higher in irg-1 than in R108 leaves (FIG. 21a). A similar trend was observed for total acids, alcohols, and alkanes in total leaf waxes extracted from leaves of an irg1-2 line.

Since SEM pictures showing a lack of epicuticular wax crystals on the abaxial surface of irg1 mutants lines correlated with changes in fungal spore germination and differentiation, it was hypothesized that the abaxial surface of irg1 might either lack a particular wax constituent that promotes germ-tube differentiation, or the mutant might accumulate an inhibitory wax constituent. Thus, epicuticular waxes from abaxial and adaxial surfaces were also isolated separately. Abaxial and adaxial leaf surface epicuticular waxes were isolated using a polymer film of gum arabic as described (Gniwotta et al., 2005). Polymer films peeled from 2-3 leaflets were pooled and extracted with hexane, evaporated and resuspended by sonication in fresh hexane.

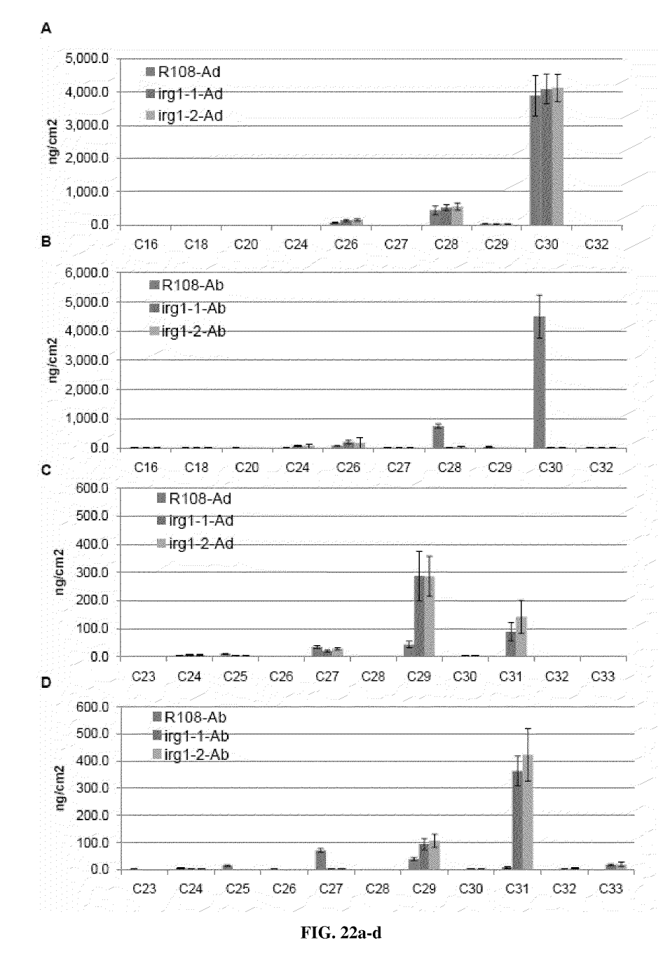

The amount of total alcohols, the predominant constituent of M. truncatula leaf waxes, and their composition were similar on the adaxial leaf surfaces of wild-type R108 and irg1 mutants (FIG. 21b; FIG. 22a-b). However, significant changes in amount and composition of alcohols and alkanes were observed when waxes isolated from abaxial surfaces of R108 and irg1 were compared (FIG. 21c; FIG. 22). Total alcohols in abaxial axes were ˜17 fold lower in irg1 mutants than in R108. Dramatic reductions in C₂₈ and C₃₀ alcohols in irg1 mutants were the major contributors for the reduction in leaf wax alcohol content (FIG. 22a). Total alkanes in abaxial wax of irg1 leaves were ˜3-fold higher than in R108 (FIG. 21c). C₂₉ and C₃₁ alkanes were found in higher amounts on both abaxial and adaxial surfaces of irg1 leaves when compared to R108. These results demonstrate that a loss of function mutation in IRG1/PALM1 leads to dramatic alterations in the amount and composition of leaf waxes.

### Example 12

### Surface Wax/Hydrophobicity is Required for Appressorium Formation by Phakopsora pachyrhizi and Puccinia emaculata

Cytological and chemical analyses described above demonstrate that irg1 mutants are defective in formation of epicuticular wax crystals and accumulation of hydrophobic alcohols on abaxial surfaces of leaves (MANU FIGS. 6-7). The hypothesis that waxy/hydrophobic surfaces and/or certain plant-derived chemical signals are required for P. pachyrhizi and P. emaculata to form pre-infection structures was therefore tested. Quantitative analyses of fungal development (spore germination, germ-tube elongation, and appressorium differentiation) were carried out on hydrophilic (glass) surfaces coated with or without epicuticular waxes isolated from wild-type M. truncatula or irg1 mutant plants. Analyses were also performed on leaf surfaces from which waxes had been manually removed.

The chloroform solution of waxes from wild-type R108 or irg1 mutants was adjusted to a final concentration of 0.05 mL/cm² leaf area. Uncoated frosted glass slides (25×75 mm, VWR International, West Chester Pa., USA) were coated 3 times with a chloroform solution to cover the whole surface. Hexane was allowed to completely evaporate between each application as described (Podila et al., 1993b). For removal of epicuticular waxes from leaves of switchgrass and soybean, detached leaf surfaces were gently rubbed with a cotton swab saturated with a solution containing bentonite (0.02% w/v, Sigma-Aldrich, St. Louis, Mo., USA) and celite (1% w/v, Sigma-Aldrich) as described (Xia et al., 2009). Urediniospores of P. emaculata and P. pachyrhizi were inoculated on respective host leaf surfaces by spotting 10 μl aliquots of a solution containing 1×10⁴ spores/ml in 0.01% Tween 20 on untreated leaf surfaces, or on leaf surfaces manipulated as described to remove waxes. The percentage of pre-infection structures were evaluated as described above.

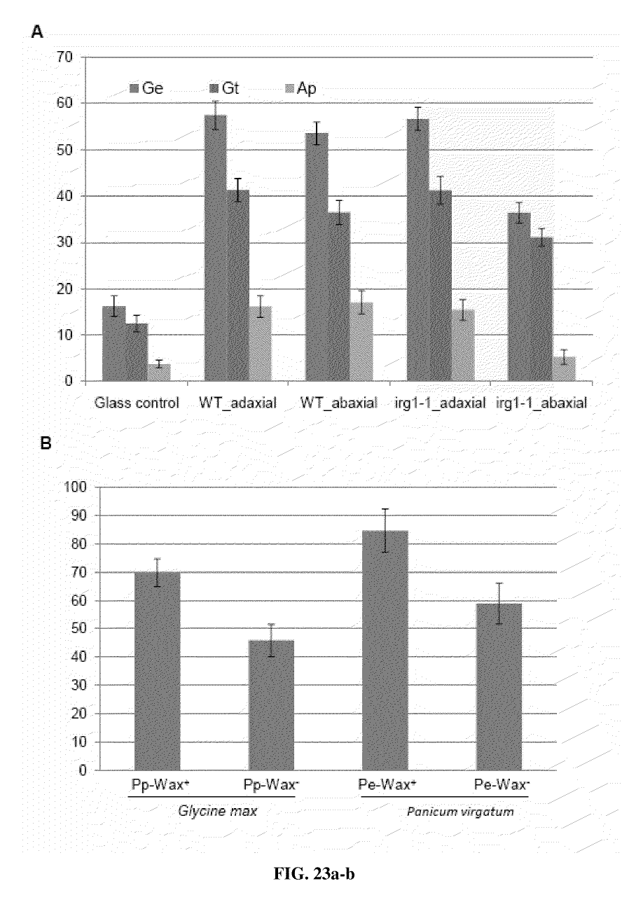

On hydrophilic (uncoated) glass surfaces, germination and differentiation of P. pachyrhizi urediniospores were severely impaired. Only 16.2% (±2.2%) of the urediniospores germinated, and less than 5% of total urediniospores spotted on glass slides formed appressoria (FIG. 23a). On glass slides coated with total waxes isolated from the abaxial or adaxial surfaces of wild-type R108, or adaxial surface of irg1, more than 50% of the P. pachyrhizi urediniospores germinated and 12-15% of total spotted urediniospores formed appressoria (FIG. 23a). On glass slides coated with total waxes isolated from abaxial surfaces of irg1 leaves, ˜30% of P. pachyrhizi urediniospores were found to germinate, but the percentage of total spores that formed appressoria was only ˜5% and was comparable to the result from the uncoated glass slide (FIG. 23a, control). These results show that abaxial waxes from irg1-1 fail to allow for appressoria formation and suggests a requirement of contact surface hydrophobicity for efficient germination and appressorium formation by P. pachyrhizi.

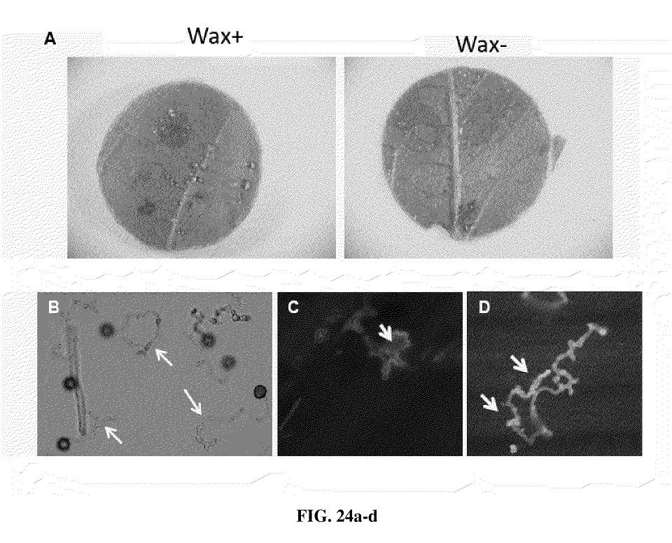

To further confirm these results, development of P. pachyrhizi urediniospores was also studied on native (“wax +”) abaxial surfaces of the host plant, soybean, and on the abaxial surface of soybean leaves that had been gently rubbed with a buffer solution containing celite and bentonite to remove the epicuticular wax layer (“wax −”). On native abaxial host surfaces, ˜70% of P. pachyrhizi urediniospores formed appressoria and were able to penetrate host epidermal cells (FIG. 23b). In contrast, ˜45% of inoculated spores formed appressoria and penetrated the epidermal layer on the manipulated host “wax −” surface. Consistent with these spore differentiation defects, a significant reduction in ASR infection was observed in detached soybean leaf surfaces manipulated to remove the surface waxes (FIG. 24a). Taken together with the glass slide experiments, the detached leaf experiments further confirm that hydrophobicity is critical for germination and differentiation of appressoria by P. pachyrhizi urediniospores.

Unlike P. pachyrhizi spores, urediniospores of P. emaculata germinated efficiently and formed long germ-tubes on hydrophilic (glass) surfaces (FIG. 24b). However, P. emaculata spores failed to form appressoria on uncoated glass slides, or on glass slides coated with waxes isolated from leaf surfaces. A possible requirement for surface waxes (hydrophobicity) for pre-penetration development (i.e. germination, germ-tube development, and appressorium differentiation) of P. emaculata urediniospores on native “wax +” abaxial surface of the host plant, switchgrass, and on “wax −” abaxial switchgrass leaf surfaces was also examined. A 35-40% reduction in appressoria formation on stomata was observed on leaf surfaces that were rubbed with the buffer solution containing celite and bentonite to remove the abaxial wax layer. (FIG. 23b). On native surfaces of switchgrass, germinated spores formed appressoria over stomatal openings (FIG. 24c). On surfaces manipulated with the buffer solution containing celite and bentonite to remove the abaxial epicuticular wax layer, although the germinated spores oriented to recognize the stomata, a significant number of them failed to form appressoria on the stomata (FIG. 24d). Thus, P. emaculata spores utilize surface signals for appressorium formation but not for initial germ-tube growth. Therefore the inhibition of P. emaculata germ-tube growth observed in irg1 mutants may not be related to reduced surface hydrophobicity.

### Example 13

### Transcript Profiling Identifies a Role of IRG1/PALM1 in Regulating Expression of Genes Involved in Long-Chain Fatty Acid Biosynthesis and Transport

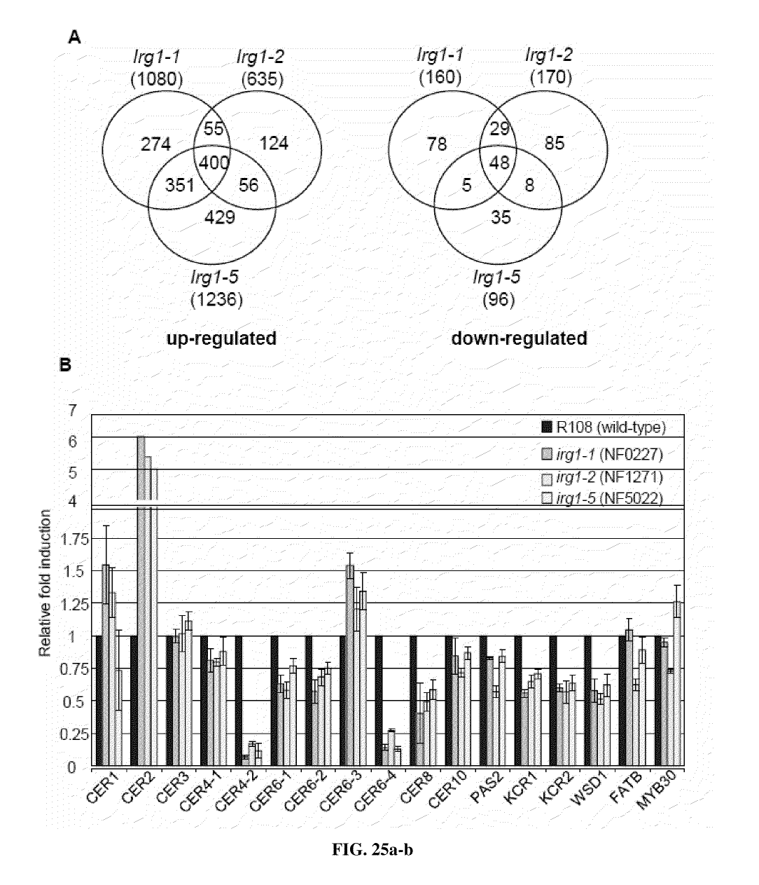

To gain a molecular understanding of how loss-of-function mutation in a gene encoding a transcription factor involved in leaf morphogenesis impacts symmetric cuticular wax deposition on a plant leaf surface, transcript profiles of M. truncatula R108 wild-type were compared with transcript profiles of three independent irg1/palm1 homozygous null mutant lines (irg1-1, irg1-2, and irg1-5; FIG. 25a; Table 5). A set of 400 commonly up-regulated and 48 commonly down-regulated genes were compared in each of the mutant lines and the wild-type, to identify the major pathways targeted by IRG1/PALM1, and genes listed in Table 5 represent a subset of these.

TABLE 5
Selected wax biosynthesis, P450, and pathogenesis-related
genes differentially regulated in irg1 mutant lines (irg1-1, irg1-2, and irg1-5). Numbers in
rightmost three columns represent fold-change in expression relative the control.

| | | irg1-1/ | irg1-2/ | irg1-5/ |
|---|---|---|---|---|
| Affy ID | Target Description | wt | wt | wt |
| Lipid metabolism | | | | |
| Mtr.9322.1.S1_at | Fatty acid elongase-like protein (Cer2) | 7.67 | 8.02 | 6.33 |
| Mtr.34695.1.S1_at | Lipid transfer protein-like protein | 5.58 | 5.32 | 8.57 |
| Mtr.35178.1.S1_at | Alpha keto-acid dehydrogenase | 4.64 | 5.29 | 3.88 |
| Mtr.40882.1.S1_at | Aldehyde dehydrogenase | 4.89 | 2.50 | 2.90 |
| Mtr.13594.1.S1_s_at | Lipase-like protein | 2.97 | 2.97 | 3.82 |
| Mtr.43284.1.S1_at | Alcohol dehydrogenase | 3.36 | 2.19 | 3.85 |
| Mtr.41915.1.S1_at | Phospholipase D alpha 1 (PLD alpha 1) | 3.14 | 3.02 | 3.20 |
| Mtr.11022.1.S1_at | RING zinc finger like protein- | 2.74 | 3.20 | 2.96 |
| Mtr.34695.1.S1_s_at | Lipid transfer protein-like protein | 2.81 | 2.84 | 3.03 |
| Mtr.13293.1.S1_at | Lipid transfer protein, partial (90%) | 2.08 | 2.58 | 2.85 |
| Mtr.42509.1.S1_at | Arabidopsis thaliana gl1 homolog (LTP) | 5.22 | 3.46 | 4.36 |
| Mtr.12797.1.S1_at | Family II lipase (EXL3) | 0.35 | 0.36 | 0.47 |
| Mtr.20073.1.S1_at | Plant lipid transfer | 0.28 | 0.40 | 0.42 |
| Mtr.26036.1.S1_sat | FA elongase 3-ketoacyl-CoA synthase 1 | 0.40 | 0.32 | 0.28 |
| Mtr.11073.1.S1_at | Short-chain dehydrogenase | 0.22 | 0.25 | 0.26 |
| Mtr.34634.1.S1_at | Epoxide hydrolase-like protein | 3.78 | 2.40 | 4.58 |
| TFs, P450s | | | | |
| Mtr.6071.1.S1_s_at | MYB96 transcription factor | 0.34 | 0.37 | 0.43 |
| Mtr.8828.1.S1_at | Cytochrome P450 | 3.29 | 2.14 | 5.98 |
| Mtr.10175.1.S1_at | Cytochrome P450 71A24 | 4.36 | 2.52 | 4.52 |
| Mtr.9388.1.S1_at | WRKY transcription factor 23 | 2.84 | 3.11 | 2.96 |
| Mtr.1299.1.S1_s_at | Cytochrome P450 93B1 | 7.10 | 2.98 | 8.91 |
| Mtr.10175.1.S1_at | Cytochrome P450 71A24 | 4.36 | 2.52 | 4.52 |
| Mtr.8987.1.S1_at | Cytochrome P450 monooxygenase | 4.76 | 2.67 | 3.74 |
| Mtr.42955.1.S1_at | Cytochrome P450 76C4 | 2.84 | 2.26 | 3.84 |
| Others | | | | |
| Mtr.331.1.Sl_at | Chitinase | 13.92 | 3.88 | 13.78 |
| Mtr.7638.1.S1_at | Endo-1,3-beta-glucanase | 5.62 | 3.07 | 5.69 |
| Mtr.7638.1.S1_sat | Endo-1,3-beta-glucanase | 5.17 | 2.75 | 4.70 |
| Mtr.12525.1.S1_at | Chitinase, complete | 4.46 | 2.54 | 4.44 |
| Mtr.39139.1.S1_at | Pathogenesis-related protein 4A | 3.61 | 2.09 | 5.53 |
| Mtr.33212.1.S1_s_at | Beta-glucosidase | 7.26 | 2.87 | 6.11 |
| Mtr.7638.1.S1_at | Endo-1,3-beta-glucanase | 5.62 | 3.07 | 5.69 |
| Mtr.39322.1.Sl_at | UDP-glucuronosyltransferase | 4.24 | 3.20 | 2.19 |

For microarray experiments, total RNA was purified from leaf tissues of four week old M. truncatula R108 and irg1 mutants using TRIzol reagent (Invitrogen, Carlsbad, Calif.). according to the manufacturer's directions. Total RNA was extracted from three independent seedlings per treatment and pooled to represent one biological replicate. Three independent pools were used to represent three biological replicates. The integrity of the RNA was confirmed on an Agilent Bioanalyzer 2100 (Agilent, Santa Clara, Calif., USA) and 10 mg of total RNA was used as a template for amplification. Probe labeling, chip hybridization, and scanning were performed according to the manufacturer's instructions (Affymetrix, Santa Clara, Calif., USA). Three biological replicates per treatment were hybridized independently to the Affymetrix GeneChip® Medicago Genome Array representing 50,900 M. truncatula genes. Raw data was imported into Robust Multi-chip Average (“RMA”) software and normalized as described (Irizarry et al., 2003). The presence/absence call for each probe set was obtained from dCHIP (Li and Wong, 2001) and genes that were differentially expressed between sample pairs were selected using Associative Analyses as described (Dozmorov and Centola, 2003). Type I family-wise error rate was reduced using Bonferroni-corrected P-value threshold of 0.5/N where N represents the number of genes on the chip. The false discovery rate was monitored and controlled by calculating the Q-value (false discovery rate) using extraction of differential gene expression (Storey and Tibshirani, 2003; Leek et al., 2006). Genes that showed significant differences in transcript levels (two-fold or greater and P-value≦9.82318×10⁻⁷) between sample pairs were selected for further analyses.

For RT-PCR experiments, qRT-PCR was performed as described (Uppalapati et al., 2009) using gene specific primers designed as based on target sequences (e.g. see Table 6 for representative primer sequences; additional primers may be designed in view of sequences known in the art). A list of corresponding Medicago orthologs of Arabidopsis wax biosynthesis-related genes, expression of which was studied using RT-qPCR and microarray analysis is found in Table 7.

TABLE 6
Primers utilized to identify changes in expression of corresponding
Medicago orthologs of Arabidopsis wax biosynthesis related genes
using RT-qPCR and microarray analysis (SEQ ID NOs: 97-132).

| Gene | Mt TC ID | Forward Primer | Reverse Primer |
|---|---|---|---|
| CER1 | MtCER1-1 (TC130292) | GCTTCTACGATAATGGCATCAAGGC | TGCTGTGAATCACCCAAGGAGCTA |
| CER2 | MtCER2 (TC115187) | AAGTGTGGTGGGATTTCATTGGGC | TCAACCCGTTTAACTGTAGCCGGA |
| CER3/WAX2 | MtCER3 (TC125004) | TCTGCGATCCGCTTCTTCATTTGC | TACTGAAAGCCACATCGCGGTACA |
| CER4 | MtCER4-1 (TC122585) | TGGGTTGAGGGTCTCAGAACCATT | ATTTGCATGAGCCACCATAGCCAC |
| | MtCER4-2 (TC160800) | TTTCCCTGGTTGGGTTGAAGGAGT | ACCACCATATCAGCAGGGATCACA |
| CER6 | MtCER6-1 (TC116151) | AGCAGCAGTTCTCCTCTCCAACAA | TCTTGAAAGACGCAGCCGTAGGAT |
| | MtCER6-2 (TC125487) | CACCTGTTACATGTCGTGTCCCTT | CCTCTTGCTGATTCCATGGTTGGT |
| | MtCER6-3 (TC121408) | TGATCCACACCGTCCGAACACATA | CTCCGGCAACCGCCATTAAATCTT |
| | MtCER6-4 (TC113573) | TCCTCTGATCGAACCCGTTCCAAA | CAAAGCATCTCCTGCAACAGCCAT |
| CER8 | MtCER8-1 (TC140235) | AAGCAAGGCTAGGTGGACGTGTTA | ATGGCGGAGTTCCAAGAGGATTGT |
| CER10 | MtCER10 (TC125186) | GGGCTTCAACATTGCAACGCAAAC | TCACTTCAATGGCTTGGGCTCCTA |
| PAS2 | MtPAS2 (GE348322) | TGCATCAGGATGCCGAATACATGG | CTTTGCTTTGGCGAGGGCTTTCTT |
| KCR1 | MtKCR1 (TC113588) | AGAAGCCCTCTTAAGCTTGAGGCA | TAAGGATAAGCCACACCAGCACCA |
| KCR2 | MtKCR2 (TC145787) | TGGGATTGATGTGCAGTGTCAGGT | AAGGGTATGTGGCCAGTATGGTGT |
| WSD1 | MtWSD1 (TC145247) | TGTTCAAGTGAAGGTGGTGGTGAG | CTTGCTGGTGAACCTAGGATGCTT |
| FATB | MtFATB (TC148248) | ATTGGCTGGATTCTGGAGAGTGCT | GTCGAAGCAAATGCTGGCACTCAA |
| MYB30 | MtMYB30 (TC142663) | AGTCTTTGTCACCAGACGCAACGA | TGAGCATGATCACCACCACAACCT |
| Ubiquitin | MtUbq | CTGACAGCCCACTGAATTGTGA | TTTTGGCATTGCTGCAAGC |

TABLE 7
List of corresponding Medicago orthologs of Arabidopsis wax biosynthesis-related genes, expression
of which was studied using RT-qPCR and microarray analysis

| | | | | | Fold |
|---|---|---|---|---|---|
| | | | | | change |
| Gene | Gene Description | At Gene ID | Mt TC ID | Affy ID | (irg1/R108) |
| CER1 | Unknown | AT1G02205 | MtCER1-1 | Mtr.37919.1.S1_at | 2.88 |
| | | | (TC130292) | | |
| CER2 | Unknown | AT4G24510 | MtCER2 (TC115187) | Mtr.9322.1.S1_at | 7.33 |
| CER3/WAX2 | Unknown | AT5G57800 | MtCER3 (TC125004) | Mtr.44147.1.S1_at | 1.04 |
| CER5 | ABC transporter | AT1G51500 | MtCER5-1 | Mtr.45091.1.S1_x_at | 1.29 |
| | | | (TC145141) | | |
| CER6 | β-keto acyl-CoA synthase (KCS) | AT1G68530 | MtCER6-4 | Mtr.26036.1.S1_s_at | 0.33 |
| | | | (TC113573) | | |
| CER8 | Long chain acyl-CoA synthase | AT2G47240 | MtCER8-1 | Mtr.32057.1.S1_at | 0.70 |
| | (LACS) | | (TC140235) | | |
| CER10 | Enoyl-CoA reductase (ECR) | AT3G55360 | MtCER10 | Mtr.38679.1.S1_at | 0.86 |
| | | | (TC125186) | | |
| KCR1 | β-keto acyl-CoA reductase | AT1G67730 | MtKCR1-2 | Mtr.48911.1.S1_at | 0.78 |
| | (KCR) | | (TC113588) | | |
| WSD1 | Wax synthase | AT5G37300 | MtWSD1 | Mtr.15935.1.S1_at | 0.82 |
| | | | (TC145247) | | |
| FATB | Fatty acyl-ACP thioesterase B | AT1G08510 | MtFATB (TC148248) | Mtr.40760.1.S1_at | 0.82 |
| | (FATB) | | | | |
| MYB30 | MYB Transcription factor | AT3G28910 | MtMYB30 | Mtr.44930.1.S1_s_at | 1.52 |
| | | | (TC142663) | | |

Total RNA was treated with Turbo DNAse (Ambion) to eliminate genomic DNA, and 5 μg of DNAse treated RNA was reverse transcribed using Superscript III™ (Invitrogen) with oligo d(T)₂₀ primers. The cDNA (1:10) was then used for qRT-PCR, which was performed using Power SYBR® Green PCR master mix (Applied Biosystems, Foster City, Calif., USA) in an optical 384-well plate with an ABI Prism 7900 HT sequence detection system (Applied Biosystems). Melt-curve analysis was performed to monitor primer-dimer formation and to check amplification of gene-specific products. The average threshold cycles (C_{T}) values calculated from triplicate biological samples were used to determine the expression level relative to controls. Primers specific for ubiquitin were used to normalize small differences in template amounts.

In irg1/palm1 lines, a gene involved in cuticular wax biosynthesis, fatty acid elongase-like protein (CER2) and genes encoding lipid transfer proteins (LTPs) were up-regulated (more than 5-fold) compared to wild-type, while no obvious down-regulation (more than two-fold) was noted (Table 5). The transcriptome data also identified genes encoding several P450 genes and pathogenesis-related proteins such as chitinases and β-1-3-glucanases that were preferentially up-regulated in irg1/palm1 mutant lines. Expression of M. truncatula orthologs of Arabidopsis genes implicated in wax biosynthesis was also determined using RT-PCR (MANU FIG. 9B; SUPPL TABLE 2). Consistent with the microarray results, significant up-regulation of CER2 (up 7-fold) was observed in irg1/palm1 (FIG. 25b; Table 7). Several other genes implicated in wax biosynthesis including CER4, CER6, CER8, β-keto acyl-CoA reductase (KCR1, KCR2); and wax synthase (WSD1) were also down-regulated. Approximately 4-fold down-regulation was observed for CER4-2 and CER6-4 genes, while others were moderately down-regulated (1.5-2 fold) in irg1/palm1 when compared to R108 wild-type (FIG. 25b; Table 7). Thus, the transcript profiles of lines with three independent irg1/palm1 alleles showed alteration in expression of genes involved in wax biosynthesis and transport.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,461,648; U.S. Pat. No. 4,535,060; U.S. Pat. No. 5,000,000; U.S. Pat. No. 5,037,663; U.S. Pat. No. 5,302,523; U.S. Pat. No. 5,322,783; U.S. Pat. No. 5,384,253; U.S. Pat. No. 5,464,765; U.S. Pat. No. 5,508,184; U.S. Pat. No. 5,538,877; U.S. Pat. No. 5,538,880; U.S. Pat. No. 5,545,818; U.S. Pat. No. 5,550,318; U.S. Pat. No. 5,563,055; U.S. Pat. No. 5,591,616; U.S. Pat. No. 5,610,042.
U.S. Pat. Publ 20040049802
Barkoulas et al., Nat Genet., 2008.
Benedito et al., Plant J., 55:504-513, 2008.
Berger et al., Development, 136:823-832, 2009.
Bharathan et al., Science, 296:1858-1860, 2002.
Blein et al., Science, 322:1835-1839, 2008.
BromWeld et al., Crop Sci., 20:254-255, 1980.
Bouton, Curr. Opin. Genetics and Develop.; 17:553-558, 2007.
Champagne et al., Plant Cell, 19:3369-3378, 2007.
Chen et al., PNAS 107:10754-10759, 2010.
Chuck et al., Plant Cell, 8:1277-1289, 1996.
Coen et al., Cell, 63:1311-1322, 1990.
David-Schwartz et al., Plant Cell, 21:3093-3104, 2009.
D'Erfurth et al., Plant J., 34:95-106, 2003.
Di Giacomo et al., Plant Mol. Biol., 67:135-150, 2008.
Dozmorov and Centola, Bioinformatics 19:204-211, 2003.
Englbrecht et al., BMC Genomics, 5:39, 2004.
Ge et al., Plant Signaling & Behavior 5:1-4, 2010.
Gniwotta et al., Pl. Physiol. 139:519-530, 2005.
Goellner et al., Mol. Plant Pathol., 11:169-77, 2010.
Graham et al., Plant Physiol., 131:872-877, 2003.
Gustafson et al., Crop Science, 43:755-759, 2003.
Hansjakob et al., New Phytol. 188:1039-1054, 2010.
Hareven et al.; Cell, 84:735-744, 1996.
Hay et al., Curr. Opin. Plant Biol., 12:593-598, 2009.
Heath Physiol. Plant Pathol. 10:73-76, 1977.
Heath, Curr. Opin. Plant Biol., 3:315-319, 2000.
Hartwig, Crop Sci., 23:237-239, 1983.
Hartwig, Crop Sci., 26:1135-1136, 1986.
Hofer et al.; Curr. Biol., 7:581-587, 1997.
Hofer et al., Plant Mol. Biol., 45:387-398, 2001.
Irizarry et al., NAR 31:e15, 2003.
Janssen et al., Plant Physiol., 117:771-786, 1998.
Koch and Hoppe, J. Phytopathol. 122:232-244, 1988.
Koenig et al., Development, 136:2997-3006, 2009.
Kolattukudy et al., PNAS, 92:4080-4087, 1995.
Kunst and Samuels, Prog. Lipid Res. 42:51-80, 2003.
Laxmi et al., PLoS One 3,:e1510, 2008.
Leek et al., Bioinformatics 22:507-508, 2006.
Li and Wong, PNAS 98:31-36, 2001.
Lincoln et al., Plant Cell, 6:1859-1876, 1994.
Long et al., Nature, 379:66-69, 1996.
Lynch et al., Plant Dis., 90:970, 2006.
McLean et al., Aust. J. Agric. Res., 31:951-956, 1980.
Miles et al., Plant Management Network, 2006.
Mysore et al., Trends Plant Sci.; 9:97-104, 2004.
Ohta et al.; Plant Cell, 13:1959-1968, 2001.
Parnis et al., Plant Cell, 9:2143-2158, 1997.
PCT App. WO 94/09699
PCT App. WO 95/06128
PCT App. WO 97/41228
PCT App. WO 97/4103
PCT App. WO 92/17598
Podila et al., Plant Physiol. 103:267-272, 1993.
Posada-Buitrago et al., Fungal Genet. Biol., 42:949-962, 2005.
Ramirez et al.; Plant Physiol., 151:1878-1888, 2009.
Sambrook et al., In: Molecular Cloning—A Laboratory Manual (second edition), Cold Spring Harbour Laboratory Press, 1989.
Samuels et al., Annu. Rev. Plant Biol. 59:698-707, 2008.
Scholte et al., Mol. Breeding, 10:203-215, 2002.
Shani et al.; Plant Cell, 21:3078-3092, 2009.
Sinha et al., Genes Dev., 7:787-795, 1993.
Storey and Tibshirani, PNAS 100:9440-9445, 2003.
Tadege et al., Plant J., 54:335-347, 2008.
Tadepally et al., BMC Evol. Biol., 8:176, 2008.
Takatsuji, Cell Mol. Life Sci., 54:582-596, 1998.
Uppalapati, Studies on Host-Pathogen Interactions between Porphyra spp. (Rhodophyta, Bangiales) and Pythium porphyrae (Oomycota, Peronosporales):104, 1999.
Uppalapati et al., Molecular Plant Microbe Intera., 22:7-17, 2009.
Van de Mortel et al., Mol. Plant Microbe Interact., 20:887-99, 2007.
Wang et al., Plant Physiol., 146:1759-1772, 2008.
Xia et al., Cell Host Microbe 5:151-155, 2009.
Young et al., Curr. Opin. Plant Biol., 12:193-201, 2009.
Zhang et al., Plant J. 42:689-707, 2005.

What is claimed is:

1. A plant comprising a down-regulated PALM transcription factor, wherein the plant exhibits an enhanced agronomic property.

2. The plant of claim 1, wherein the plant comprises a mutated genomic PALM gene or a DNA molecule capable of expressing a nucleic acid sequence complementary to all or a portion of a PALM messenger RNA (mRNA).

3. The plant of claim 2, wherein the plant comprises a DNA molecule that when transcribed produces a nucleic acid sequence complementary to all or a portion of a PALM mRNA.

4. The plant of claim 3, wherein the nucleic acid sequence complementary to all or a portion of a PALM mRNA comprises a sequence complementary to all or a portion of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95.

5. The plant of claim 2, wherein the plant comprises a mutated genomic PALM gene.

6. The plant of claim 5, wherein the mutated genomic PALM gene comprises a deletion, a point mutation or an insertion in a wild-type PALM gene.

7. The plant of claim 5, wherein the mutated genomic PALM gene is produced by irradiation, T-DNA insertion, transposon insertion or chemical mutagenesis.

8. The plant of claim 1, wherein the plant is forage plant, a biofuel crop, or a legume.

9. The plant of claim 8, wherein the forage plant is a forage soybean, alfalfa, clover, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass or reed canarygrass.

10. The plant of claim 8, wherein the biofuel crop is switchgrass (Panicum virgatum), giant reed (Arundo donax), reed canarygrass (Phalaris arundinacea), Miscanthus×giganteus, Miscanthus sp., sericea lespedeza (Lespedeza cuneata), corn, sugarcane, sorghum, millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass or poplar.

11. The plant of claim 8, wherein the legume is soybean.

12. The plant of claim 1, wherein the enhanced agronomic property is selected from the group consisting of increased forage nutritional content, increased disease resistance and increased leafy tissue content.

13. The plant of claim 1, wherein the enhanced agronomic property is increased leaf to stem ratio.

14. The plant of claim 1, wherein the enhanced agronomic property is enhanced resistance to a fungal pathogen.

15. The plant of claim 14, wherein the fungal pathogen is a rust pathogen.

16. The plant of claim 14, wherein the fungal pathogen is P. emaculata, P. pachyrhizi or Colletotrichum trifolii.

17. The plant of claim 3, wherein the DNA molecule comprises a nucleic acid sequence complementary to all or a portion of a PALM mRNA operably linked to a promoter sequence selected from the group consisting of a developmentally-regulated, organelle-specific, inducible, tissue-specific, constitutive, cell-specific, seed specific, or germination-specific promoter.

18. The plant of claim 1, further defined as an R₀ transgenic plant.

19. The plant of claim 1, further defined as a progeny plant of any generation of an R0 transgenic plant, wherein the transgenic plant has inherited the selected DNA from the R0 transgenic plant.

20. The plant of claim 1, wherein the PALM transcription factor comprises a sequence selected from the group consisting of SEQ ID NO: 70; SEQ ID NO: 72; SEQ ID NO: 74; SEQ ID NO: 76; SEQ ID NO: 78; SEQ ID NO: 80; SEQ ID NO: 82; SEQ ID NO: 84; SEQ ID NO: 86; SEQ ID NO: 88; SEQ ID NO: 90; SEQ ID NO: 92; SEQ ID NO: 94; and SEQ ID NO: 96.

21. A seed that produces the plant of claim 1.

22. A plant part of the plant of claim 1.

23. The plant part of claim 22, further defined a protoplast, cell, meristem, root, leaf, pistil, anther, flower, seed, embryo, stalk or petiole.

24. A nucleic acid molecule comprising a nucleic acid sequence selected from the group consisting of:
(a) a nucleic acid sequence that hybridizes to the nucleic acid sequence complementary to the sequence of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95, under conditions of 1×SSC and 65° C.;
(b) a nucleic acid comprising the sequence complementary to SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95 or a fragment thereof; and
(c) a nucleic acid sequence exhibiting at least 80% sequence identity to a complement of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95;
wherein the nucleic acid sequence is operably linked to a heterologous promoter sequence and wherein expression of the nucleic acid molecule in a plant cell down-regulates a PALM transcription factor.

25. The nucleic acid molecule of claim 24, wherein the DNA molecule comprises a nucleic acid sequence exhibiting at least 85%, 90%, 95%, 96%, 97%, 98% or 99% sequence identity to a complement of SEQ ID NO: 69; SEQ ID NO: 71; SEQ ID NO: 73; SEQ ID NO: 75; SEQ ID NO: 77; SEQ ID NO: 79; SEQ ID NO: 81; SEQ ID NO: 83; SEQ ID NO: 85; SEQ ID NO: 87; SEQ ID NO: 89; SEQ ID NO: 91; SEQ ID NO: 93; or SEQ ID NO: 95.

26. The nucleic acid molecule of claim 24, wherein the heterologous promoter sequence is a developmentally-regulated, organelle-specific, inducible, tissue-specific, constitutive, cell-specific, seed specific, or germination-specific promoter.

27. A transgenic plant cell comprising the nucleic acid molecule of claim 24.

28. A transgenic plant or plant part comprising the nucleic acid molecule of claim 24.

29. A biofuel feedstock comprising a nucleic acid molecule of claim 24.

30. A method of conferring at least a first altered agronomic property to a plant comprising down-regulating a PALM transcription factor in said plant.

31. The method of claim 30, wherein the altered agronomic property is increased nutritional content of a forage crop plant.

32. The method of claim 30, wherein the altered agronomic property is increased digestibility of a forage crop plant.

33. The method of claim 30, wherein the altered agronomic property is increased disease resistance.

34. The method of claim 33, wherein the disease is caused by a fungus.

35. The method of claim 34, wherein the disease is caused by a rust fungus.

36. The method of claim 34, wherein the fungus is P. emaculata, P. pachyrhizi or Colletotrichum trifolii.

37. The method of claim 34, wherein the fungus forms a pre-infection structure on the plant at a reduced frequency as compared with the frequency of formation of a pre-infection structure on an otherwise isogenic plant in which a PALM transcription factor is not down-regulated.

38. The method of claim 37, wherein the pre-infection structure comprises a germ-tube or an appressorium.

39. The method of claim 30, wherein the altered agronomic property is altered epicuticular wax content.

40. A method for producing a commercial product comprising obtaining a plant of claim 1 or a part thereof and producing a commercial product therefrom.

41. The method of claim 40, wherein the commercial product is ethanol, biodiesel, silage, animal feed or fermentable biofuel feedstock.


SEQUENCE LISTING (132 sequences)

SEQ ID NO: 1 - DNA, 39 bp, Artificial sequence
  Synthetic primer
acgacgttgtaaaacgaccgagccaattttgttagacga

SEQ ID NO: 2 - DNA, 25 bp, Artificial sequence
  Synthetic primer
tcaaaaactttattttaggcatcca

SEQ ID NO: 3 - DNA, 20 bp, Artificial sequence
  Synthetic primer
ggtttctttgggatcaagca

SEQ ID NO: 4 - DNA, 20 bp, Artificial sequence
  Synthetic primer
aaaccgcagcaaagaaaaga

SEQ ID NO: 5 - DNA, 20 bp, Artificial sequence
  Synthetic primer
gcacttgtgtgcaacattga

SEQ ID NO: 6 - DNA, 20 bp, Artificial sequence
  Synthetic primer
tcgcgttcatttaaaacgtg

SEQ ID NO: 7 - DNA, 20 bp, Artificial sequence
  Synthetic primer
tggatgccacaccctctatt

SEQ ID NO: 8 - DNA, 20 bp, Artificial sequence
  Synthetic primer
gttgggggtgtcaaatatcg

SEQ ID NO: 9 - DNA, 20 bp, Artificial sequence
  Synthetic primer
ccatacaaagaagcgggtgt

SEQ ID NO: 10 - DNA, 20 bp, Artificial sequence
  Synthetic primer
aaactgtttggctcgcttgt

SEQ ID NO: 11 - DNA, 19 bp, Artificial sequence
  Synthetic primer
gtgctttccccctcaaaaa

SEQ ID NO: 12 - DNA, 21 bp, Artificial sequence
  Synthetic primer
gatagctgctggattggaaca

SEQ ID NO: 13 - DNA, 20 bp, Artificial sequence
  Synthetic primer
tgacttcccacctcatcctc

SEQ ID NO: 14 - DNA, 20 bp, Artificial sequence
  Synthetic primer
tacattcccctggaatttgg

SEQ ID NO: 15 - DNA, 20 bp, Artificial sequence
  Synthetic primer
gtggcagtacccctgtctgt

SEQ ID NO: 16 - DNA, 20 bp, Artificial sequence
  Synthetic primer
ggtgcaatggtaaggttgct

SEQ ID NO: 17 - DNA, 20 bp, Artificial sequence
  Synthetic primer
atcaatgacatggacccaca

SEQ ID NO: 18 - DNA, 20 bp, Artificial sequence
  Synthetic primer
catccctttggctgacctaa

SEQ ID NO: 19 - DNA, 20 bp, Artificial sequence
  Synthetic primer
tgcccaaatgtgtttccata

SEQ ID NO: 20 - DNA, 20 bp, Artificial sequence
  Synthetic primer
aatttcatggcttgggtttg

SEQ ID NO: 21 - DNA, 20 bp, Artificial sequence
  Synthetic primer
ttgtctctcgaatggtgtgg

SEQ ID NO: 22 - DNA, 20 bp, Artificial sequence
  Synthetic primer
cgatcatgcatggtttgaag

SEQ ID NO: 23 - DNA, 33 bp, Artificial sequence
  Synthetic primer
tcatgaattctgcaatattattattatttaatg

SEQ ID NO: 24 - DNA, 37 bp, Artificial sequence
  Synthetic primer
ttaatctagaggccagcgtacttatctcttcctatac

SEQ ID NO: 25 - DNA, 24 bp, Artificial sequence
  Synthetic primer
ccatggctacagatattggccttc

SEQ ID NO: 26 - DNA, 30 bp, Artificial sequence
  Synthetic primer
ggttacctcaagttggtgttggcttgttcc

SEQ ID NO: 27 - DNA, 28 bp, Artificial sequence
  Synthetic primer
aaaggatccatggctacagatattggcc

SEQ ID NO: 28 - DNA, 25 bp, Artificial sequence
  Synthetic primer
cccctcgagagttggtgttggcttg

SEQ ID NO: 29 - DNA, 28 bp, Artificial sequence
  Synthetic primer
tttctcgagatggctacagatattggcc

SEQ ID NO: 30 - DNA, 29 bp, Artificial sequence
  Synthetic primer
tttccatggctcaagttggtgttggcttg

SEQ ID NO: 31 - DNA, 27 bp, Artificial sequence
  Synthetic primer
aagtactctttatcatgaattctgcaa

SEQ ID NO: 32 - DNA, 22 bp, Artificial sequence
  Synthetic primer
gaaggcacaatccagcattagc

SEQ ID NO: 33 - DNA, 18 bp, Artificial sequence
  Synthetic primer
ccacctctccgtccccaa

SEQ ID NO: 34 - DNA, 22 bp, Artificial sequence
  Synthetic primer
cagcgtgctcactgtaaaacca

SEQ ID NO: 35 - DNA, 22 bp, Artificial sequence
  Synthetic primer
cccaaccaccgttaaattcttc

SEQ ID NO: 36 - DNA, 22 bp, Artificial sequence
  Synthetic primer
gaaggcacaatccagcattagc

SEQ ID NO: 37 - DNA, 22 bp, Artificial sequence
  Synthetic primer
tcaatgtgcctgccatgtatgt

SEQ ID NO: 38 - DNA, 22 bp, Artificial sequence
  Synthetic primer
actcacaccgtcaccagaatcc

SEQ ID NO: 39 - DNA, 23 bp, Artificial sequence
  Synthetic primer
tgagcacgctcttcttgctttca

SEQ ID NO: 40 - DNA, 24 bp, Artificial sequence
  Synthetic primer
ggtggtggcatccatcttgttaca

SEQ ID NO: 41 - DNA, 21 bp, Artificial sequence
  Synthetic primer
ttggactgccaaaagattgga

SEQ ID NO: 42 - DNA, 17 bp, Artificial sequence
  Synthetic primer
ccgtgccgccgtaattc

SEQ ID NO: 43 - DNA, 18 bp, Artificial sequence
  Synthetic primer
ccccaacccgtgaaatca

SEQ ID NO: 44 - DNA, 19 bp, Artificial sequence
  Synthetic primer
cgcgatccagactgaatgc

SEQ ID NO: 45 - DNA, 22 bp, Artificial sequence
  Synthetic primer
caccatggctacagatattggc

SEQ ID NO: 46 - DNA, 22 bp, Artificial sequence
  Synthetic primer
tcaagttggtgttggcttgttc

SEQ ID NO: 47 - DNA, 22 bp, Artificial sequence
  Synthetic primer
atggaagaataccagcatgaca

SEQ ID NO: 48 - DNA, 21 bp, Artificial sequence
  Synthetic primer
ttatggaccgagacgataagg

SEQ ID NO: 49 - DNA, 20 bp, Artificial sequence
  Synthetic primer
ccgtaccgagccgcaggaac

SEQ ID NO: 50 - DNA, 23 bp, Artificial sequence
  Synthetic primer
cagatctcggtgacgggcaggac

SEQ ID NO: 51 - DNA, 27 bp, Artificial sequence
  Synthetic primer
ctccagacatttttatttttcaccaag

SEQ ID NO: 52 - DNA, 27 bp, Artificial sequence
  Synthetic primer
gcattcaaactagaagacagtgctacc

SEQ ID NO: 53 - DNA, 20 bp, Artificial Sequence
  Synthetic primer
tatggtagctcatgtgttgg

SEQ ID NO: 54 - DNA, 20 bp, Artificial sequence
  Synthetic primer
tgaagaaaggtagatggcag

SEQ ID NO: 55 - DNA, 20 bp, Artificial sequence
  Synthetic primer
aggcaataaggaaaagtagc

SEQ ID NO: 56 - DNA, 20 bp, Artificial sequence
  Synthetic primer
acccataataatatccgacc

SEQ ID NO: 57 - DNA, 20 bp, Artificial sequence
  Synthetic primer
aaccacgtctatctatagcc

SEQ ID NO: 58 - DNA, 22 bp, Artificial sequence
  Synthetic primer
tttggaaaattatgagaagtgg

SEQ ID NO: 59 - DNA, 20 bp, Artificial sequence
  Synthetic primer
aggcaataaggaaaagtagc

SEQ ID NO: 60 - DNA, 20 bp, Artificial sequence
  Synthetic primer
cctctgatttgacttgactg

SEQ ID NO: 61 - DNA, 20 bp, Artificial sequence
  Synthetic primer
aggcaataaggaaaagtagc

SEQ ID NO: 62 - DNA, 21 bp, Artificial sequence
  Synthetic primer
aattgatgctttgggttgtcg

SEQ ID NO: 63 - DNA, 20 bp, Artificial sequence
  Synthetic primer
aggcaataaggaaaagtagc

SEQ ID NO: 64 - DNA, 23 bp, Artificial sequence
  Synthetic primer
agggttatttagttcaaatgttc

SEQ ID NO: 65 - DNA, 20 bp, Artificial sequence
  Synthetic primer
aggcaataaggaaaagtagc

SEQ ID NO: 66 - DNA, 20 bp, Artificial sequence
  Synthetic primer
tggtaaggtcctggtcagtg

SEQ ID NO: 67 - DNA, 20 bp, Artificial sequence
  Synthetic primer
aaccacgtctatctatagcc

SEQ ID NO: 68 - DNA, 21 bp, Artificial sequence
  Synthetic primer
tagtgttattcccaagactgg

SEQ ID NO: 69 - DNA, 756 bp, Medicago truncatula
atggctacagatattggccttctttccaatatgactcagatccaaaaatcatcacaatcacaatctcaacaacaccaaccaaaccctaattccaacaccactacacccccctcaccatcttcatcaacttggatgtggaaccctaaacaacaccaagaacaagaagatgaagattcatgggaggtaagggcttttgctgaagacacaaggaatattatgaacacaacatggccaccaaggtcctacacctgtaccttttgcagaagagagttccggtcagctcaagctcttggtggtcacatgaatgttcaccgccgcgaccgtgctcgtctccatcaaacccaaccaccgttaaattcttctcatccttcatcaccattcataaatattcctccacaagaccttgttgctaatgctggattgtgccttctttaccatttaccaaaccctaataataatgcttttgcttccttcaatagttctagtcctaatggagaatctccttctacttttctctcaatctcatcatcaacatcttatcctccaaataatttgatgatgcaaatgcaagcttgttctccatcttttaattttcaagtggataattcagctaggttgatcaacaatagcatttcttctttttctagcaaagtggaccagcaacatgctacttgcacctccattgatgataatggtcatgaaattgaagagcttgatcttgagctccgtttagggaacaagccaacaccaacttga

SEQ ID NO: 70 - protein, 251 aa, Medicago truncatula
MATDIGLLSNMTQIQKSSQSQSQQHQPNPNSNTTTPPSPSSSTWMWNPKQHQEQEDEDSWEVRAFAEDTRNIMNTTWPPRSYTCTFCRREFRSAQALGGHMNVHRRDRARLHQTQPPLNSSHPSSPFINIPPQDLVANAGLCLLYHLPNPNNNAFASFNSSSPNGESPSTFLSISSSTSYPPNNLMMQMQACSPSFNFQVDNSARLINNSISSFSSKVDQQHATCTSIDDNGHEIEELDLELRLGNKPTPT

SEQ ID NO: 71 - DNA, 765 bp, Medicago Sativa
atggctacagatattggccttctttccaatatgactcagatccaaaaatcatcacaatcacaatctcaacaacaccaaccaaaccctaactccaacaccactacaaccccttcaccatcatcatcaacttggatgtggaaccctaaacaacaccaacagcaacaagaacaagaagatgaagattcatgggaggtaagggcttttgctgaagacacaaggaatattatgaacacaacatggccaccaaggtcttatacctgcactttttgcagaagagagttccggtcagctcaagctcttggtggtcacatgaatgttcaccgccgcgaccgtgctcgtctccatcaaagccaaccaccgttaaattcatctcatccttcatcaccattcataaatattcctccacaagagcttgttgctaatgctggattgtgccttctttaccatttaccaaaccctaataataatgcttttgcttcctacaatagttctagtcctaatggagaatctccttctacttttctctcaatctcatcatcaacatcttatcctccaaataatttgatgatgcaaatgcaaacttgttctccatcttttaattttcaagtggataattcagctaggttgatcaataatagcatttcttctttttctagcaaagtggaccatcaacatgctacttgcacctccattgatgataacggtcatgaaattgaagagcttgatcttgagctccgtttagggaacaagccaacaccaacttga

SEQ ID NO: 72 - protein, 254 aa, Medicago sativa
MATDIGLLSNMTQIQKSSQSQSQQHQPNPNSNTTTTPSPSSSTWMWNPKQHQQQQEQEDEDSWEVRAFAEDTRNIMNTTWPPRSYTCTFCRREFRSAQALGGHMNVHRRDRARLHQSQPPLNSSHPSSPFINIPPQELVANAGLCLLYHLPNPNNNAFASYNSSSPNGESPSTFLSISSSTSYPPNNLMMQMQTCSPSFNFQVDNSARLINNSISSFSSKVDHQHATCTSIDDNGHEIEELDLELRLGNKPTPT

SEQ ID NO: 73 - DNA, 624 bp, Glycine max
atgtggaaccctagggagcaacaacagcagcaggtagaagatgatgatgactcctgggaggtcagagcttttgcagaagacacaaggaacatcatgggcaccacatggcctcctaggtcctacacctgcaccttctgcagaagggagtttcgctccgcccaagccctcggcggccacatgaacgtccaccgccgcgaccgggcccgcctccaccaagctccaccttcctcctgctccaaccccatgtcctcttctcttcccacttcatccttcatcaatatccctcctcaagagcttgttggaaatgctggcttgtgcctcctctaccacttgcccacgagccctagtagtgcaccacccttcccttcacccacttctaatggggcttctccctccactcttctctctatctcctcctatcctacaaacaactttctgatgcaaacttcctttaattttcccggtgcacccccaactgggatcaatactactactgtttcctctttgtgctataactctagcaaagtggaacaatccgcaacttcttcctctattgataatggccacctcgaagagcttgatctagagctccgcctcgggcacaaaccaacaccaacctcataa

SEQ ID NO: 74 - protein, 207 aa, Glycine max
MWNPREQQQQQVEDDDDSWEVRAFAEDTRNIMGTTWPPRSYTCTFCRREFRSAQALGGHMNVHRRDRARLHQAPPSSCSNPMSSSLPTSSFINIPPQELVGNAGLCLLYHLPTSPSSAPPFPSPTSNGASPSTLLSISSYPTNNFLMQTSFNFPGAPPTGINTTTVSSLCYNSSKVEQSATSSSIDNGHLEELDLELRLGHKPTPTS

SEQ ID NO: 75 - DNA, 619 bp, Glycine max
atgtggaaccctaggaagcaacaacatcagcagcagcaggtagaagatgatgatgactcttgggaggtcagagcttttgcagaagacacaaggaacatcatgggcaccacatggcctcctaggtcctacacctgcaccttctgcagaagggagtttcgctccgcccaagccctcggcggccacatgaacgtccaccgccgcgaccgcgctcgcctccaccaagctccagtaccctcctcctcctccaaccccatgacctcttctcttcccacttcacccttcatcaatatccctcctcaagaccttgttgcaaatgctggcttgtgcctcctctaccacttgcccaaaagccctagtagtactgcaccacccttcccaccttcccccactactccttctccttccactcttctctctatctcttcttatcctacaaccaactttctgatgcaaacttcctttaattttcccggtgcacccgcaactgggatcaatactactactactactactgtttcctctttgtgctataactctagcaaagtggaacaatccgcaacttcctcctctattgatcataatggccacctcgaagagcttgatctagagctccgcctag

SEQ ID NO: 76 - protein, 206 aa, Glycine max
MWNPRKQQHQQQQVEDDDDSWEVRAFAEDTRNIMGTTWPPRSYTCTFCRREFRSAQALGGHMNVHRRDRARLHQAPVPSSSSNPMTSSLPTSPFINIPPQDLVANAGLCLLYHLPKSPSSTAPPFPPSPTTPSPSTLLSISSYPTTNFLMQTSFNFPGAPATGINTTTTTTVSSLCYNSSKVEQSATSSSIDHNGHLEELDLELRL

SEQ ID NO: 77 - DNA, 726 bp, Lotus japonicus
atgactcagatccaaaaattatcaccatcaccatcaacatcatcacaacatccccaaccaaaccctaactccgcctctaccaccaccaccacgacctccgccacttggatgtggaaccctagcagcagagagaaccaccgtgaggaagctgaagaagatgagtcatgggaggtcagagccttcgccgaagacacaaggaacatcatgggaaccacatggcctccgaggtcctacacctgcaccttctgcagaagggagttccgttcagcccaagctctcggcggccacatgaatgtccaccgccgtgaccgcgctcgcctccaccaagttcacccgcccagttccatcccctctcaccctacaccacccttcataaacattccacctcatcaccaggaccttggtttgtgccttctctatcatttgccaagccctaatagtactactgttcccaatcttaatgtaaatggagaatctccctctactcttctctccatctcaccctatcaaccaaacaacttgatgatgcaaacttgctcttcaccttcatttaattttccactagctcaggtttcttcacctcatatcaatggcagtgttccttcttacttctgcaactccaccaccaaactggagcaacctccaaccttaagatctattgataatggccacctggaagagcttgatctagagctccgtctcgggcacaaggcaacaccaacatga

SEQ ID NO: 78 - protein, 241 aa, Lotus japonicus
MTQIQKLSPSPSTSSQHPQPNPNSASTTTTTTSATWMWNPSSRENHREEAEEDESWEVRAFAEDTRNIMGTTWPPRSYTCTFCRREFRSAQALGGHMNVHRRDRARLHQVHPPSSIPSHPTPPFINIPPHHQDLGLCLLYHLPSPNSTTVPNLNVNGESPSTLLSISPYQPNNLMMQTCSSPSFNFPLAQVSSPHINGSVPSYFCNSTTKLEQPPTLRSIDNGHLEELDLELRLGHKATPT

SEQ ID NO: 79 - DNA, 615 bp, Arabidopsis thaliana
atgaacggtggtgcatggatgtggaaccctaacaaaattgaagaattggaggatgatgatgaatcttgggaagtcaaagcctttgagcaagacactaaaggcaacatctctggtaccacttggcctccaagatcttacacttgcaatttctgccgccgtgagttccgttctgctcaagccttaggcggtcacatgaatgtccaccgccgtgaccgcgcctcatctagggctcatcaaggttccaccgttgcggctgcggctagaagcggccacggggggatgttactcaattcttgtgctccgccgttgcctacaacgacacttataatacaatccacggcgagtaacattgaaggtttgtcccatttctaccaactgcaaaaccctagtggcatttttggtaattctggtgacatggtgaatctttatggtacgacttcgtttcctccgagcaaccttccgttttcaatgttgaattctccagtagaagttcctcctcggcttattgaatattcgacaggagatgatgagagcattggctcgatgaaagaagcgacaggaacatcagtggatgagcttgatcttgaacttcggctagggcaccatccaccgtga

SEQ ID NO: 80 - protein, 204 aa, Arabidopsis thaliana
MNGGAWMWNPNKIEELEDDDESWEVKAFEQDTKGNISGTTWPPRSYTCNFCRREFRSAQALGGHMNVHRRDRASSRAHQGSTVAAAARSGHGGMLLNSCAPPLPTTTLIIQSTASNIEGLSHFYQLQNPSGIFGNSGDMVNLYGTTSFPPSNLPFSMLNSPVEVPPRLIEYSTGDDESIGSMKEATGTSVDELDLELRLGHHPP

SEQ ID NO: 81 - DNA, 753 bp, Vitis vinifera
atggctgctgagcttggccttgtgtccttgacccagctccagaaattggctcagtctcagcagcatcaacgcccggaggaagaaaaccctagctcaacaagcagctcgtggatgtggaaccctaagcaagcacagacacaagcacaagcacaagcacaagaagatgatgattcatgggaggtaagagcctttgcagaagacactggcaatatcatgggcaccacttggccacctaggtcatacacttgcactttctgtagaagggaattccgttctgcccaagccctaggaggtcacatgaatgtgcaccgccgcgaccgagcgaggctccaccaaacccacccgggttcaaacaaccccacttcatcatcctccactaactcatcatccaccctcataatcccaacccaagaattcatcacaaatggtgggctatgccttctctaccagctaccacctaaccctaatgccctcttcacccctacttccataaattcatgtatggattccccctccactctcctctccatcgccccgtatcctcccaataacttgatctcaccttgccctccatcaattaatttttcagcaccaccacaaggcatcgctctctgtccctccagcaaacccgagccgtctgcagtgtccaatggcgacgacgataatgagaataccagcaacaattacaacgactcagccatggaggagcttgatctagagctccgactaggacacagaccctcgccgtcataa

SEQ ID NO: 82 - protein, 250 aa, Vitis vinifera
MAAELGLVSLTQLQKLAQSQQHQRPEEENPSSTSSSWMWNPKQAQTQAQAQAQEDDDSWEVRAFAEDTGNIMGTTWPPRSYTCTFCRREFRSAQALGGHMNVHRRDRARLHQTHPGSNNPTSSSSTNSSSTLIIPTQEFITNGGLCLLYQLPPNPNALFTPTSINSCMDSPSTLLSIAPYPPNNLISPCPPSINFSAPPQGIALCPSSKPEPSAVSNGDDDNENTSNNYNDSAMEELDLELRLGHRPSPS

SEQ ID NO: 83 - DNA, 615 bp, Arabidopsis lyrata; identical to SEQ ID NO: 79

SEQ ID NO: 84 - protein, 204 aa, Arabidopsis lyrata; identical to SEQ ID NO: 80

SEQ ID NO: 85 - DNA, 585 bp, Cucumis sativus
atgtggaatcctaaccaagctcatcaagacgaggatgatgattcatgggagattagagcttttgcagaagacaccggaaacattatgggcacaacttggccccctaggttttataattgcacattttgcggacgagaatttagatctgcccaagccctaggtggtcacatgaatgtccaccgtcgcgaccgtgtacgtttccatcaccaaatccaacccaactcaattcaacccatctcaccgtctttcaccatccctacccctaaactcatctacaatgaaattgacgaggtttgtttcttataccaactaccgaacgacaacatcaatttcctcaactccatcacttcatcagattcatgtctccagtcatccttcactgcacaacatccttcgagcactcgaaccgcgtcgtccttgcaatccctaaagtctccaggagagcttcgaggtggaacgtcgagctcctcgtctcactgcagccacatatctagcaaaggagatgactcattaatatcaattaatgatggaaatgagaaggtggatcttgagttaagacttggtcatagggcatctccaacttga

SEQ ID NO: 86 - protein, 194 aa, Cucumis sativus
MWNPNQAHQDEDDDSWEIRAFAEDTGNIMGTTWPPRFYNCTFCGREFRSAQALGGHMNVHRRDRVRFHHQIQPNSIQPISPSFTIPTPKLIYNEIDEVCFLYQLPNDNINFLNSITSSDSCLQSSFTAQHPSSTRTASSLQSLKSPGELRGGTSSSSSHCSHISSKGDDSLISINDGNEKVDLELRLGHRASPT

SEQ ID NO: 87 - DNA, 621 bp, Manihot esculenta
atgtggaaccctaagcaaactcaagaagatgatgattcatgggaggtcagagcttttcaagaggacacaggtaatgcaatgggaaccacttggcctccacgttcttatacttgcactttttgcagaagagaatttcgttctgctcaagccctaggtggtcacatgaacgtgcaccgccgtgaccgtgctaggctccaccagacagtgcttcagccaccgcctggttcaatcaaaccacccagctcatcaacttctacttcttcatctgctatcttaatcccagctcaagaattttccactaatggtggtgggttatgtttgctctaccaattaccaaaccctaatggagttttttcttccacagctatgaatgcatgtgctgttgagtctcctactcttctctctatctcgccgtatcaccatagcaacttgattgggcaagctcttaattatccagcagcttcatcattgataaattcttctcatttttactcaagcaaaccagaatctgcagcctcttttgacaagtgcaaggagatgggaagtgaagaacttgatctagagctgaggctcggccacagatcgacgaaatcatcatcatcatcatcatccccatcatga

SEQ ID NO: 88 - protein, 206 aa, Manihot esculenta
MWNPKQTQEDDDSWEVRAFQEDTGNAMGTTWPPRSYTCTFCRREFRSAQALGGHMNVHRRDRARLHQTVLQPPPGSIKPPSSSTSTSSSAILIPAQEFSTNGGGLCLLYQLPNPNGVFSSTAMNACAVESPTLLSISPYHHSNLIGQALNYPAASSLINSSHFYSSKPESAASFDKCKEMGSEELDLELRLGHRSTKSSSSSSSPS

SEQ ID NO: 89 - DNA, 624 bp, Mimulus guttatus
atgtggagcaacaagcagacaccgcccggagatgattcatgggaagtccgggcattcgaagaagacacaaccggaaacctattaggttgcacgtggcccccacgctcctacatgtgcactttttgccggagggagtttcgctccgcgcaagctcttggcggccacatgaatgtgcaccgccgggatcgagccaggctgcacgcggaattgccgatgcccccgctaccgcctgtttcgccttctccggcaaccaccgggcaagagtttgtgggaaatggattgtgcctagtctaccctttacataaccctaacaacagtatcagtatcagtaccaccgtattgttccctgctccacccgtaccggataatttccgatctcctccccttccgatatcccatttcttcaagagtgaacccaattgtgaaaccgcaccacacagtgtcgtttcttcgctttgtcactctagcaaaaccaaaccgtcaccgtcgaacagtaacgaaaactgcgacaaaaacaaaaaccttacaaagggtgagaagaacgttgttaaagattctgctgcagcggacgccattgaagagctcgatctggaacttcgtctgggaagaagagatgagcactga

SEQ ID NO: 90 - protein, 207 aa, Mimulus guttatus
MWSNKQTPPGDDSWEVRAFEEDTTGNLLGCTWPPRSYMCTFCRREFRSAQALGGHMNVHRRDRARLHAELPMPPLPPVSPSPATTGQEFVGNGLCLVYPLHNPNNSISISTTVLFPAPPVPDNFRSPPLPISHFFKSEPNCETAPHSVVSSLCHSSKTKPSPSNSNENCDKNKNLTKGEKNVVKDSAAADAIEELDLELRLGRRDEH

SEQ ID NO: 91 - DNA, 738 bp, Populus trichocarpa
atggctgcagagattggccttctctccttgacccaactccaaaaattacctcaatctcaacaaaatcagtatcaactgcatcaactgaacccgaatgagacccctagtgtctggatgtggaaccctaagcaaactcaagaagaggatgattcatgggaggttagagccttcgcagaggataccggcaacatcaacggcaccacttggccaccgaggtcttatacttgcaccttttgtagaagggaattccgctcagctcaagccctagggggtcacatgaatgttcaccgccgtgaccgtgctaggcttcaccaaacacagcctggttcaatcaaccccaactcatcaacttccagttcttcctcgtctacttttataatcccaactcaagaatttcccccaaatgctgggttatgcttactttaccaactaccaaaccctaatggagtcttcactcccgcaactatgaatgcatgtgctactgattcaccttctactcttctctctatcacaccatatccccataacaacttgatagagaaatctcttaattttctagtagctccacctgagataaatacttctcattgttactcaatcaaagccgagccctcggcatccattgataatagcaataatatcaacagcgacaacaactttaaggagttggcacacgaagaacttgatctagagctccggctagggcacagatcgacaacaccaccaccatcatcataa

SEQ ID NO: 92 - protein, 245 aa, Populus trichocarpa
MAAEIGLLSLTQLQKLPQSQQNQYQLHQLNPNETPSVWMWNPKQTQEEDDSWEVRAFAEDTGNINGTTWPPRSYTCTFCRREFRSAQALGGHMNVHRRDRARLHQTQPGSINPNSSTSSSSSSTFIIPTQEFPPNAGLCLLYQLPNPNGVFTPATMNACATDSPSTLLSITPYPHNNLIEKSLNFLVAPPEINTSHCYSIKAEPSASIDNSNNINSDNNFKELAHEELDLELRLGHRSTTPPPSS

SEQ ID NO: 93 - DNA, 699 bp, Ricinus comunis
atggctgctgagcttggtcttctctccctagcccagctagaactccaaaaactagcggaatctcagcaaaatcagcatcaacatcaactgaactcatcaagttcttggatgtggaaccctaagcaaactcatgaagatgaagactcgtgggaggttagagcctttgaagaagatacaggaaatatcatgggcaccacttggccgccgcggtcttatacttgcaccttttgtagaagagaattcaggtcagctcaagccctaggtggtcacatgaatgtgcaccgccgggaccgtgctaggctacatcaaacaccacccggttcaatcaaccccaactcatcatcaactaattctacttccgcttccactttcataatcccagctcgagagttttctaccaatggcgggttatgcctgctctaccaattacctaaccctaacgggatattcactaccacagctatgaatgcatgtgctattgatcattcaccatctactcttctctctatctcaccctatccacataattatccggtcgcatcaccagtggtaaattcttctcatttttactctagcaaagctcaaccggtagcatctacagataatagcagcaataattgcaaggacttgggaaatgaagaacttgatctagagctcaggctagggcacagatcaacatcatcttcatcgccatcataa

SEQ ID NO: 94 - protein, 232 aa, Ricinus comunis
MAAELGLLSLAQLELQKLAESQQNQHQHQLNSSSSWMWNPKQTHEDEDSWEVRAFEEDTGNIMGTTWPPRSYTCTFCRREFRSAQALGGHMNVHRRDRARLHQTPPGSINPNSSSTNSTSASTFIIPAREFSTNGGLCLLYQLPNPNGIFTTTAMNACAIDHSPSTLLSISPYPHNYPVASPVVNSSHFYSSKAQPVASTDNSSNNCKDLGNEELDLELRLGHRSTSSSSPS

SEQ ID NO: 95 - DNA, 636 bp, Carica Papaya
atggctgctgagctcggccttctctccttgacccagctcagaaacatagctagcttatcgcccggcgcatggatgtggaaccctacgcaagtcgcggaacaagaagatgactcatgggaggttagagcttttgagcaagacacaggtaacattatgggtactacatggccaccaaggtcctacacctgcactttctgcagacgagaattccggtcagcccaagccctaggcggtcacatgaatgttcatcgccgcgaccgagcccggctccaccagagttcggctccacaaccgcccggtcgtgctgcaacttcaatgataatcccaactcaagacttaactgcaaatggcggtttgtgtcttctctaccaattgcctaaccctaaagggattttagcagctccaactggtgataccattgattcaccttcgactattctttctatctcaccctatccttctcaccacttgataaccccacctttgaattttacattgtcaccgtcatctacgtctaccaaagctcaatccccgtgggtgaagtcggctgatgtcagcgccaacagattaattacagcagatcattcagcagccatggaggaagttgatcttgagcttcgcttaggaccgtcttga

SEQ ID NO: 96 - protein, 211 aa, Carica Papaya
MAAELGLLSLTQLRNIASLSPGAWMWNPTQVAEQEDDSWEVRAFEQDTGNIMGTTWPPRSYTCTFCRREFRSAQALGGHMNVHRRDRARLHQSSAPQPPGRAATSMIIPTQDLTANGGLCLLYQLPNPKGILAAPTGDTIDSPSTILSISPYPSHHLITPPLNFTLSPSSTSTKAQSPWVKSADVSANRLITADHSAAMEEVDLELRLGPS

SEQ ID NO: 97 - DNA, 25 bp, Artificial sequence
  Synthetic primer
gcttctacgataatggcatcaaggc

SEQ ID NO: 98 - DNA, 24 bp, Artificial sequence
  Synthetic primer
tgctgtgaatcacccaaggagcta

SEQ ID NO: 99 - DNA, 24 bp, Artificial sequence
  Synthetic primer
aagtgtggtgggatttcattgggc

SEQ ID NO: 100 - DNA, 24 bp, Artificial sequence
  Synthetic primer
tcaacccgtttaactgtagccgga

SEQ ID NO: 101 - DNA, 24 bp, Artificial sequence
  Synthetic primer
tctgcgatccgcttcttcatttgc

SEQ ID NO: 102 - DNA, 24 bp, Artificial sequence
  Synthetic primer
tactgaaagccacatcgcggtaca

SEQ ID NO: 103 - DNA, 24 bp, Artificial sequence
  Synthetic primer
tgggttgagggtctcagaaccatt

SEQ ID NO: 104 - DNA, 24 bp, Artificial sequence
  Synthetic primer
atttgcatgagccaccatagccac

SEQ ID NO: 105 - DNA, 24 bp, Artificial sequence
  Synthetic primer
tttccctggttgggttgaaggagt

SEQ ID NO: 106 - DNA, 24 bp, Artificial sequence
  Synthetic primer
accaccatatcagcagggatcaca

SEQ ID NO: 107 - DNA, 24 bp, Artificial sequence
  Synthetic primer
agcagcagttctcctctccaacaa

SEQ ID NO: 108 - DNA, 24 bp, Artificial sequence
  Synthetic primer
tcttgaaagacgcagccgtaggat

SEQ ID NO: 109 - DNA, 24 bp, Artificial sequence
  Synthetic primer
cacctgttacatgtcgtgtccctt

SEQ ID NO: 110 - DNA, 24 bp, Artificial sequence
  Synthetic primer
cctcttgctgattccatggttggt

SEQ ID NO: 111 - DNA, 24 bp, Artificial sequence
  Synthetic primer
tgatccacaccgtccgaacacata

SEQ ID NO: 112 - DNA, 24 bp, Artificial sequence
  Synthetic primer
ctccggcaaccgccattaaatctt

SEQ ID NO: 113 - DNA, 24 bp, Artificial sequence
  Synthetic primer
tcctctgatcgaacccgttccaaa

SEQ ID NO: 114 - DNA, 24 bp, Artificial sequence
  Synthetic primer
caaagcatctcctgcaacagccat

SEQ ID NO: 115 - DNA, 24 bp, Artificial sequence
  Synthetic primer
aagcaaggctaggtggacgtgtta

SEQ ID NO: 116 - DNA, 24 bp, Artificial sequence
  Synthetic primer
atggcggagttccaagaggattgt

SEQ ID NO: 117 - DNA, 24 bp, Artificial sequence
  Synthetic primer
gggcttcaacattgcaacgcaaac

SEQ ID NO: 118 - DNA, 24 bp, Artificial sequence
  Synthetic primer
tcacttcaatggcttgggctccta

SEQ ID NO: 119 - DNA, 24 bp, Artificial sequence
  Synthetic primer
tgcatcaggatgccgaatacatgg

SEQ ID NO: 120 - DNA, 24 bp, Artificial sequence
  Synthetic primer
ctttgctttggcgagggctttctt

SEQ ID NO: 121 - DNA, 24 bp, Artificial sequence
  Synthetic primer
agaagccctcttaagcttgaggca

SEQ ID NO: 122 - DNA, 24 bp, Artificial sequence
  Synthetic primer
taaggataagccacaccagcacca

SEQ ID NO: 123 - DNA, 24 bp, Artificial sequence
  Synthetic primer
tgggattgatgtgcagtgtcaggt

SEQ ID NO: 124 - DNA, 24 bp, Artificial sequence
  Synthetic primer
aagggtatgtggccagtatggtgt

SEQ ID NO: 125 - DNA, 24 bp, Artificial sequence
  Synthetic primer
tgttcaagtgaaggtggtggtgag

SEQ ID NO: 126 - DNA, 24 bp, Artificial sequence
  Synthetic primer
cttgctggtgaacctaggatgctt

SEQ ID NO: 127 - DNA, 24 bp, Artificial sequence
  Synthetic primer
attggctggattctggagagtgct

SEQ ID NO: 128 - DNA, 24 bp, Artificial sequence
  Synthetic primer
gtcgaagcaaatgctggcactcaa

SEQ ID NO: 129 - DNA, 24 bp, Artificial sequence
  Synthetic primer
agtctttgtcaccagacgcaacga

SEQ ID NO: 130 - DNA, 24 bp, Artificial sequence
  Synthetic primer
tgagcatgatcaccaccacaacct

SEQ ID NO: 131 - DNA, 22 bp, Artificial sequence
  Synthetic primer
ctgacagcccactgaattgtga

SEQ ID NO: 132 - DNA, 19 bp, Artificial sequence
  Synthetic primer
ttttggcattgctgcaagc